(19) United States
(12) Patent Application Publication
(10) Pub. No.: US 2016/0051185 A1
(43) Pub. Date: Feb. 25, 2016

(54) SYSTEM AND METHOD FOR CREATING A DYNAMIC ACTIVITY PROFILE USING EARPHONES WITH BIOMETRIC SENSORS

(71) Applicant: JayBird LLC, Salt Lake City, UT (US)

(72) Inventors: BEN WISBEY, Canberra (AU); DAVID SHEPHERD, Canberra (AU); STEPHEN DUDDY, Moama (AU)

(73) Assignee: JAYBIRD LLC, Salt Lake City, UT (US)

(21) Appl. No.: 14/934,054

(22) Filed: Nov. 5, 2015

Related U.S. Application Data
(63) Continuation-in-part of application No. 14/830,549, filed on Aug. 19, 2015.
Application No. 14/830,549 is a continuation-in-part of application No. 14/568,835, filed on Dec. 12, 2014.
Application No. 14/568,835 is a continuation-in-part of application No. 14/140,414, filed on Dec. 24, 2013.
Application No. 14/140,414 is a continuation-in-part of application No. 14/137,942, filed on Dec. 20, 2013.
Application No. 14/137,942 is a continuation-in-part of application No. 14/137,734, filed on Dec. 20, 2013.
Application No. 14/137,734 is a continuation-in-part of application No. 14/062,815, filed on Oct. 24, 2013.

(51) Int. Cl. A61B 5/00; G06F 19/00; A61B 5/024

(52) U.S. Cl. CPC A61B 5/486; A61B 5/02438; A61B 5/6803; A61B 5/7271; A61B 5/742; A61B 5/4806; A61B 5/02416; G06F 19/322; A61B 2560/0475

(57) ABSTRACT

A system for creating and dynamically updating a user activity profile includes an earphone, a processor, a sensor module, a memory module. The memory module includes stored computer program code that, along with the memory module and the processor, is configured to carry out a number of operations to create and dynamically update the user activity profile. One such operation involves maintaining an activity archive that includes activity information received from the sensor module and that is representative of a user's activity. Another such operation includes creating and updating a dynamic activity profile based on initial user input and further based on the activity archive. The initial user input contributes to the dynamic activity profile according to a first weighting factor, and the activity archive contributes to the dynamic profile according to a second weighting factor.

### CROSS-REFERENCE TO RELATED APPLICATIONS

This application is a continuation-in-part of and claims the benefit of U.S. patent application Ser. No. 14/830,549 filed Aug. 19, 2015, titled “Earphones with Biometric Sensors,” the contents of which are incorporated herein by reference in their entirety. This application is also a continuation-in-part of and claims the benefit of U.S. patent application Ser. No. 14/568,835, filed Dec. 12, 2014, titled “System and Method for Creating a Dynamic Activity Profile,” which is a continuation-in-part of U.S. patent application Ser. No. 14/140,414, filed Dec. 24, 2013, titled “System and Method for Providing an Intelligent Goal Recommendation for Activity Level”; which is a continuation-in-part of U.S. patent application Ser. No. 14/137,942, filed Dec. 20, 2013, titled “System and Method for Providing an Interpreted Recovery Score”; which is a continuation-in-part of U.S. patent application Ser. No. 14/137,734, filed Dec. 20, 2013, titled “System and Method for Providing a Smart Activity Score”; which is a continuation-in-part of U.S. patent application Ser. No. 14/062,815, filed Oct. 24, 2013, titled “Wristband with Removable Activity Monitoring Device.” The contents of the Ser. No. 14/568,835 application, the Ser. No. 14/140,414 application, the Ser. No. 14/137,942 application, the Ser. No. 14/137,734 application, and the Ser. No. 14/062,815 application, are incorporated herein by reference in their entireties.

### TECHNICAL FIELD

The present disclosure relates generally to activity monitoring devices, and more particularly to systems and methods for creating a dynamic activity profile for a user based on the user's monitored activity as monitored using earphones with biometric sensors.

### BACKGROUND

Conventional activity monitoring and lifestyle/fitness tracking devices generally enable only a recommendation of activity that accounts for desired calories burned, steps taken—in other words, the tracked metrics are static, and do not adjust based on a user's actual behavior. Thus, one issue with conventional devices is that they do not learn a user's tendencies regarding activity, such as trends in the user's activity levels. Moreover, existing devices do not robustly track or capture the user's activity to include, for example, sleep activity or fatigue or recovery levels of the user. Another issue is that currently available solutions do not recommend activity levels based on an analysis of changes and trends in a user's activity profile—as such, conventional activity monitoring and fitness/lifestyle devices do not have the capability of recommending personalized, yet achievable goals for the user. Nor do conventional activity monitoring and fitness/lifestyle devices have the capability of recommending activity based on a learned, dynamic activity profile for the user. Additionally, conventional solutions lack the ability to provide activity recommendations based on an integration of a learned activity profile for a user with scheduled events and/or training load models.

### BRIEF SUMMARY OF THE DISCLOSURE

In view of the above drawbacks, there exists a long-felt need for an activity monitoring and fitness/lifestyle device and system that tracks user activity levels and analyzes and responds to multifaceted inputs regarding the user's activity. Further, there is a need for fitness monitoring/lifestyle devices that embody such systems to dynamically update a user activity profile and provide personalized, yet realistic goals for the user's activity levels based on ongoing trends and changes in the user activity profile. Activity goals and recommendations for a user may be more effective when the goals are finely tuned to the user's in flux capabilities and physiology. Such finely tuned goals may be generated based on the disclosed systems and methods for creating a dynamic user activity profile that tracks, analyzes, and integrates robust information about the user.

Embodiments of the present disclosure provide systems, methods, and apparatus for creating a dynamic user activity profile that tracks user activity according to multifaceted data points, and analyzes those data points to effectively learn the user's tendencies regarding activity. Moreover, by tracking robust datasets for a user, and by the analysis disclosed herein, the dynamic activity profile may be finely tuned to the user's in flux capabilities and physiology, such that personalized, yet realistic goals and activity recommendations may be provided to the user.

According to one embodiment of the disclosure, an apparatus for creating and dynamically updating a user activity profile includes an initial activity profile module, a sensor module, an activity archive module, and a dynamic activity profile module. The initial activity profile module creates an activity profile for a user. The sensor module monitors the user's activity to generate activity information. The activity archive module maintains an activity archive that includes the activity information. The activity information may include one or more of heart rate variability data, activity level data, sleep data, subjective feedback data, activity level data, and training load data. The dynamic activity profile module updates the activity profile based on the activity archive. In one example implementation, one or more of the initial activity profile module, the sensor module, the activity archive module, and the dynamic activity profile module are embodied in a pair of earphones with biometric sensors.

According to another embodiment, the apparatus includes an activity recommendation module that provides a recommendation related to the user's activity and based on the activity profile. The apparatus, in one instance, includes a profile accuracy module that provides an indication of an estimated level of accuracy of the activity profile. The estimated level of accuracy is based on an amount and a consistency of the activity information. In some embodiments, the indication of the estimated level of accuracy may be provided via a visible light indicator (e.g. light emitting diode) embedded within the housing of the apparatus, provided via an audio signal transmitted by the apparatus, or provided via a display of a computing device communicatively coupled to the apparatus, for example.

Another aspect of the present disclosure involves a method for creating and dynamically updating a user activity profile. The method includes creating an activity profile for a user, tracking the user's activity, and creating and updating a dynamic activity profile based on the activity profile and the user's activity. The method also includes, in one example implementation, creating and updating an activity archive based on the user's activity.

Creating the activity profile may include modifying the user input according to normative statistical data. Tracking the user's activity, in one instance, includes monitoring a movement of the user using an earphone or pair of earphones with biometric sensors. In another instance, tracking the user's activity includes determining an activity level of the user. And, in such an instance, the dynamic activity profile is based on one or more of an average of the user's activity level, a range of the user's activity level, and a skew of the user's activity level. Tracking the user's activity may also include tracking a set of activity parameters and providing an activity recommendation based on one or more of the activity parameters. The set of activity parameters, in one example implementation of the disclosure, includes heart rate variability, sleep duration, sleep quality, subjective feedback from the user, previous activity levels, and training load data.

In one embodiment, the activity profile contributes to the dynamic activity profile according to a first weighting factor, and the user's activity contributes to the dynamic activity profile according to a second weighting factor. Such an embodiment may also involve decreasing the first weighting factor as information about the user's activity is tracked and stored in the activity archive, and increasing the second weighting factor as information about the user's activity is tracked and stored in the activity archive. The method may, in one case, also include receiving user input to an activity questionnaire. In such a case, the activity profile is based on the user input to the activity questionnaire.

An additional aspect of the present disclosure includes a system for creating and dynamically updating a user activity profile. The system includes a processor, a sensor module, and a memory module. The memory module includes stored computer program code. The memory module, the stored computer program code, and the processor are configured to maintain an activity archive that includes activity information received from the sensor module and that is representative of a user's activity. Additionally, the memory module, the stored computer program code, and the processor are configured to create and update a dynamic activity profile based on initial user input and further based on the activity archive. The initial user input contributes to the dynamic activity profile according to a first weighting factor, and the activity archive contributes to the dynamic profile according to a second weighting factor.

In one embodiment of the system, the memory module, the stored computer program code, and the processor are further configured to vary the first and second weighting factors based on the activity information maintained in the activity archive. The memory module, the stored computer program code, and the processor may be further configured to recommend activities for the user based on the dynamic activity profile. In one example implementation of the system, the processor, the sensor module, and the memory module are embodied in an earphone or pair of earphones with biometric sensors.

### BRIEF DESCRIPTION OF THE DRAWINGS

The present disclosure, in accordance with one or more various embodiments, is described in detail with reference to the following figures.

FIG. 1 illustrates an example communications environment in which embodiments of the disclosed technology may be implemented.

FIG. 2A illustrates a perspective view of exemplary earphones that may be used to implement the technology disclosed herein.

FIG. 2B illustrates an example architecture for circuitry of the earphones of FIG. 2A.

FIG. 3A illustrates a perspective view of a particular embodiment of an earphone, including an optical heartrate sensor, in accordance with the disclosed technology.

FIG. 3B illustrates a side perspective view of placement of the optical heartrate sensor of the earphones of FIG. 3A when they are worn by a user.

FIG. 3C illustrates a frontal perspective view of placement of the optical heartrate sensor of the earphones of FIG. 3A when they are worn by a user.

FIG. 3D illustrates a cross-sectional view of an over-the-ear configuration of dual-fit earphones in accordance with the disclosed technology.

FIG. 3E illustrates a cross-sectional view of an over-the-ear configuration of the dual-fit earphones of FIG. 3D.

FIG. 3F illustrates a cross-sectional view of an under-the-ear configuration of the dual-fit earphones of FIG. 3D.

FIG. 4A is a block diagram illustrating an example computing device that may be used to implement embodiments of the disclosed technology.

FIG. 4B illustrates modules of an example activity monitoring application that may be used to implement embodiments of the disclosed technology.

FIG. 5 is an operational flow diagram illustrating a method of prompting a user to adjust the placement of earphones in the user's ear to ensure accurate biometric data collection by the earphones' biometric sensors.

FIG. 6 illustrates an activity display that may be associated with an activity display module of the activity monitoring application of FIG. 4B.

FIG. 7 illustrates an example system for creating a dynamic user activity profile.

FIG. 8 illustrates an example apparatus for creating a dynamic user activity profile.

FIG. 9 illustrates another example apparatus for creating a dynamic user activity profile.

FIG. 10 is an operation flow diagram illustrating an example method for creating a dynamic user activity profile.

FIG. 11 is an operational flow diagram illustrating another example method for creating a dynamic user activity profile.

FIG. 12 illustrates a sleep display that may be associated with a sleep display module of the activity monitoring application of FIG. 4B.

FIG. 13 illustrates an activity recommendation and fatigue level display that may be associated with an activity recommendation and fatigue level display module of the activity monitoring application of FIG. 4B.

FIG. 14 illustrates a biological data and intensity recommendation display that may be associated with a biological data and intensity recommendation display module of the activity monitoring application of FIG. 4B.

FIG. 15 illustrates an example computing module that may be used to implement various features of the systems and methods disclosed herein.

The figures are provided for purposes of illustration only and merely depict typical or example embodiments of the disclosure. The figures are described in greater detail in the description and examples below, and are not intended to be exhaustive or to limit the disclosure to the precise form disclosed. It should be understood that the disclosure may be practiced with modification or alteration, and that the disclosure may be limited only by the claims and the equivalents thereof.

### DETAILED DESCRIPTION

The present disclosure is directed to various embodiments of systems and methods for creating a dynamic user activity profile. The details of some example embodiments of the systems, methods, and apparatus of the present disclosure are set forth in the description below. Other features, objects, and advantages of the disclosure will be apparent to one of skill in the art upon examination of the present description, figures, examples, and claims. It is intended that all such additional systems, methods, features, and advantages, etc., be included within this description, be within the scope of the present disclosure, and be protected by one or more of the accompanying claims.

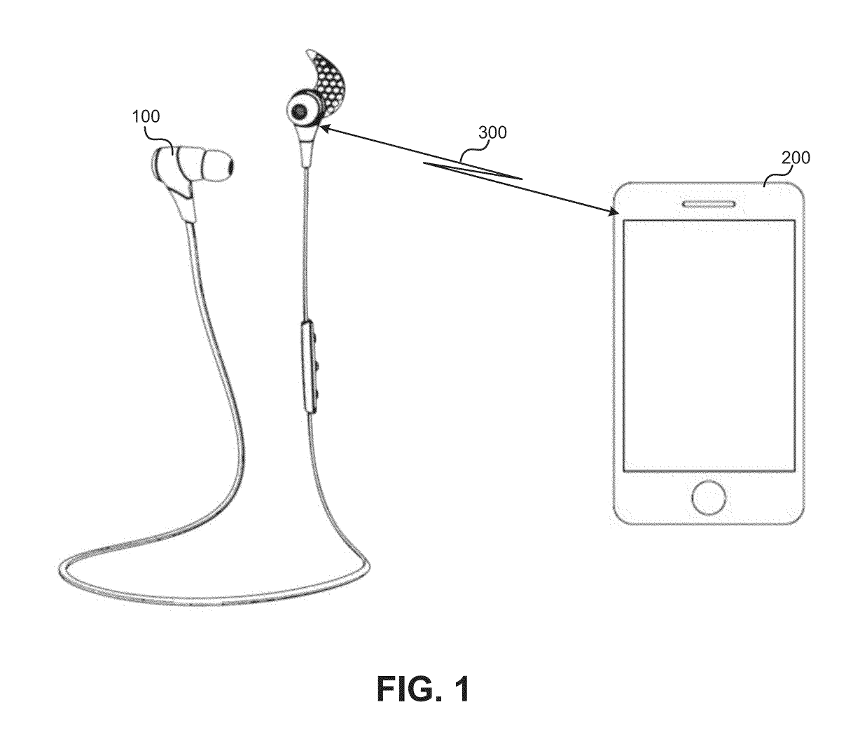

Various embodiments of the disclosed systems, methods, and apparatus for creating a dynamic user activity profile are implemented in conjunction with a wearable device, configured to be convenient for on-the-go applications and to capture a user's activity in such applications, as well as in other applications. In some example implementations, one or more biometric sensors (e.g. heartrate sensor, motion sensor, etc.) are coupled to a device that is attachable to a user—for example, the attachable device may be in the form of an earphone or a pair of earphones (used interchangeably throughout this disclosure) having biometric sensors coupled thereto, and/or including an activity monitoring module. In some embodiments, such biometric earphones may be further configured with electronic components and circuitry for processing detected user biometric data and providing user biometric data to another computing device (e.g. smartphone, laptop, desktop, tablet, etc.). FIGS. 1-6 illustrate, by way of example, embodiments that utilize such biometric earphones. Because such an attachable device provides context for the disclosed systems and methods for creating a dynamic user activity profile, various examples of the device will be described with reference to FIGS. 1 through 6. It should also be noted, however, that the disclosed systems, methods, and apparatus may be implemented using any mobile or handheld device (e.g., smartphone) in communication with biometric earphones 100, whether or not such mobile device is wearable or attachable to a user.

FIG. 1 illustrates an example communications environment in accordance with an embodiment of the technology disclosed herein. In this embodiment, earphones 100 communicate biometric and audio data with computing device 200 over a communication link 300. The biometric data is measured by one or more sensors (e.g., heart rate sensor, accelerometer, gyroscope) of earphones 100. Although a smartphone is illustrated, computing device 200 may comprise any computing device (smartphone, tablet, laptop, smartwatch, desktop, etc.) configured to transmit audio data to earphones 100, receive biometric data from earphones 100 (e.g., heartrate and motion data), and process the biometric data collected by earphones 100. In additional embodiments, computing device 200 itself may collect additional biometric information that is provided for display. For example, if computing device 200 is a smartphone it may use built in accelerometers, gyroscopes, and a GPS to collect additional biometric data.

Computing device 200 additionally includes a graphical user interface (GUI) to perform functions such as accepting user input and displaying processed biometric data to the user. The GUI may be provided by various operating systems known in the art, such as, for example, iOS, Android, Windows Mobile, Windows, Mac OS, Chrome OS, Linux, Unix, a gaming platform OS, etc. The biometric information displayed to the user can include, for example a summary of the user's activities, a summary of the user's fitness levels, activity recommendations for the day, the user's heart rate and heart rate variability (HRV), and other activity related information. User input that can be accepted on the GUI can include inputs for interacting with an activity tracking application further described below.

In embodiments, the communication link 300 is a wireless communication link based on one or more wireless communication protocols such as BLUETOOTH, ZIGBEE, 602.11 protocols, Infrared (IR), Radio Frequency (RF), etc. Alternatively, the communications link 300 may be a wired link (e.g., using any one or a combination of an audio cable, a USB cable, etc.)

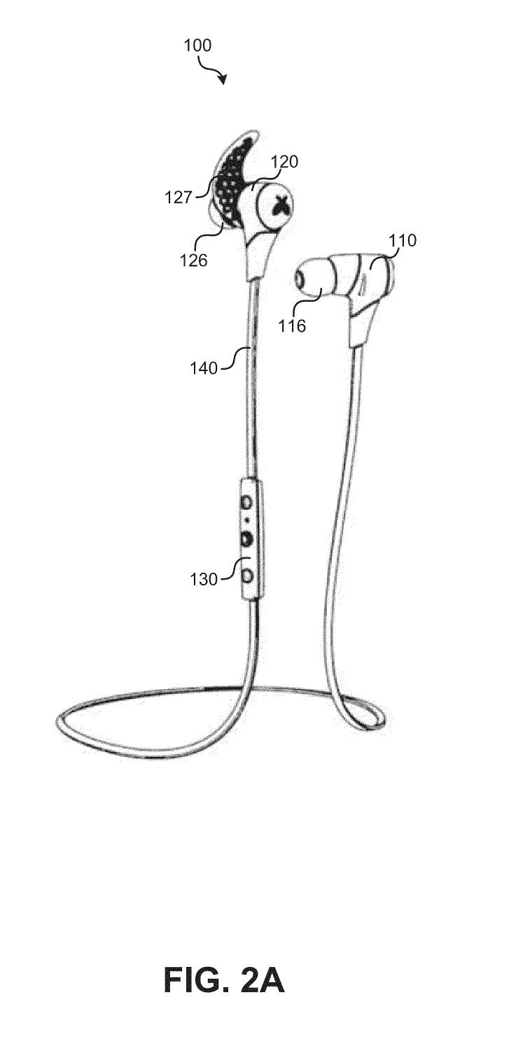

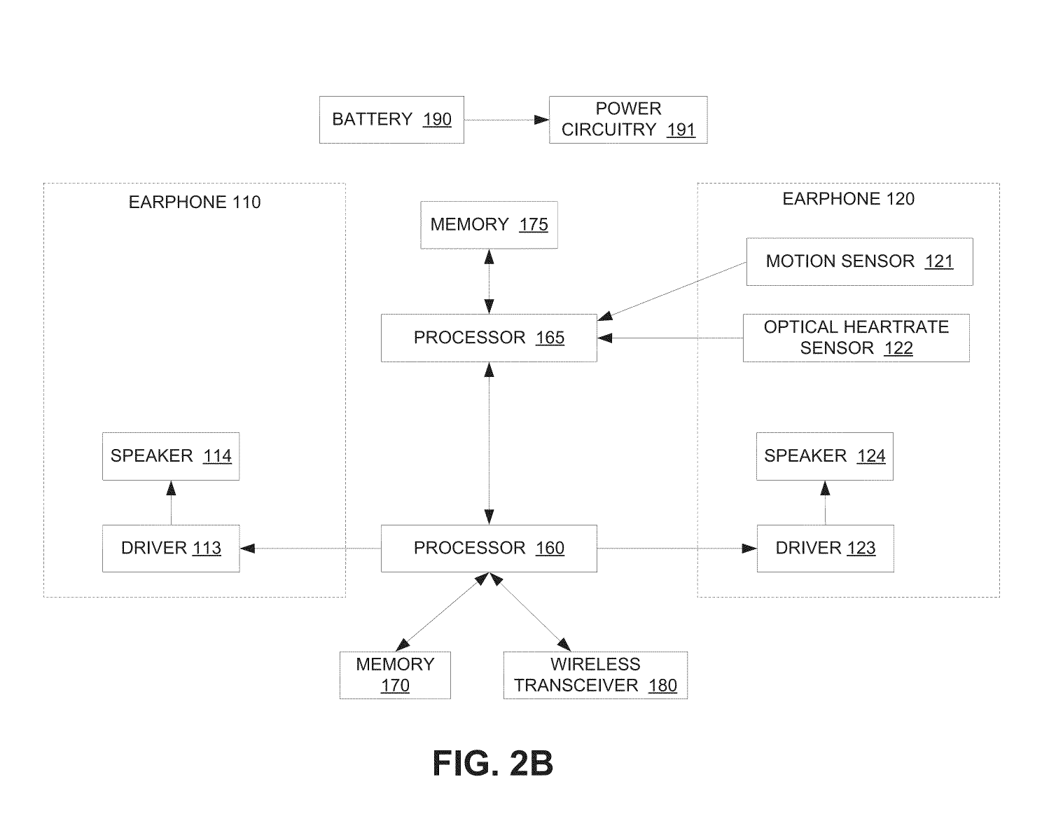

With specific reference now to earphones 100, FIG. 2A is a diagram illustrating a perspective view of exemplary earphones 100. FIG. 2A will be described in conjunction with FIG. 2B, which is a diagram illustrating an example architecture for circuitry of earphones 100. Earphones 100 comprise a left earphone 110 with tip 116, a right earphone 120 with tip 126, a controller 130 and a cable 140. Cable 140 electrically couples the left earphone 110 to the right earphone 120, and both earphones 110-120 to controller 130. Additionally, each earphone may optionally include a fin or ear cushion 127 that contacts folds in the outer ear anatomy to further secure the earphone to the wearer's ear.

In embodiments, earphones 100 may be constructed with different dimensions, including different diameters, widths, and thicknesses, in order to accommodate different human ear sizes and different preferences. In some embodiments of earphones 100, the housing of each earphone 110, 120 is rigid shell that surrounds electronic components. For example, the electronic components may include motion sensor 121, optical heartrate sensor 122, audio-electronic components such as drivers 113, 123 and speakers 114, 124, and other circuitry (e.g., processors 160, 165, and memories 170, 175). The rigid shell may be made with plastic, metal, rubber, or other materials known in the art. The housing may be cubic shaped, prism shaped, tubular shaped, cylindrical shaped, or otherwise shaped to house the electronic components.

The tips 116, 126 may be shaped to be rounded, parabolic, and/or semi-spherical, such that it comfortably and securely fits within a wearer's ear, with the distal end of the tip contacting an outer rim of the wearer's outer ear canal. In some embodiments, the tip may be removable such that it may be exchanged with alternate tips of varying dimensions, colors, or designs to accommodate a wearer's preference and/or fit more closely match the radial profile of the wearer's outer ear canal. The tip may be made with softer materials such as rubber, silicone, fabric, or other materials as would be appreciated by one of ordinary skill in the art.

In embodiments, controller 130 may provide various controls (e.g., buttons and switches) related to audio playback, such as, for example, volume adjustment, track skipping, audio track pausing, and the like. Additionally, controller 130 may include various controls related to biometric data gathering, such as, for example, controls for enabling or disabling heart rate and motion detection. In a particular embodiment, controller 130 may be a three button controller.

The circuitry of earphones 100 includes processors 160 and 165, memories 170 and 175, wireless transceiver 180, circuity for earphone 110 and earphone 120, and a battery 190. In this embodiment, earphone 120 includes a motion sensor 121 (e.g., an accelerometer or gyroscope), an optical heartrate sensor 122, and a speaker 124 and corresponding driver 123. Earphone 110 includes a speaker 114 and corresponding driver 113. In additional embodiments, earphone 110 may also include a motion sensor (e.g., an accelerometer or gyroscope), and/or an optical heartrate sensor.

A biometric processor 165 comprises logical circuits dedicated to receiving, processing and storing biometric information collected by the biometric sensors of the earphones. More particularly, as illustrated in FIG. 2B, processor 165 is electrically coupled to motion sensor 121 and optical heartrate sensor 122, and receives and processes electrical signals generated by these sensors. These processed electrical signals represent biometric information such as the earphone wearer's motion and heartrate. Processor 165 may store the processed signals as biometric data in memory 175, which may be subsequently made available to a computing device using wireless transceiver 180. In some embodiments, sufficient memory is provided to store biometric data for transmission to a computing device for further processing.

During operation, optical heartrate sensor 122 uses a photoplethysmogram (PPG) to optically obtain the user's heart rate. In one embodiment, optical heartrate sensor 122 includes a pulse oximeter that detects blood oxygenation level changes as changes in coloration at the surface of a user's skin. More particularly, in this embodiment, the optical heartrate sensor 122 illuminates the skin of the user's ear with a light-emitting diode (LED). The light penetrates through the epidermal layers of the skin to underlying blood vessels. A portion of the light is absorbed and a portion is reflected back. The light reflected back through the skin of the user's ear is then obtained with a receiver (e.g., a photodiode) and used to determine changes in the user's blood oxygen saturation (SpO2) and pulse rate, thereby permitting calculation of the user's heart rate using algorithms known in the art (e.g., using processor 165). In this embodiment, the optical sensor may be positioned on one of the earphones such that it is proximal to the interior side of a user's tragus when the earphones are worn.

In various embodiments, optical heartrate sensor 122 may also be used to estimate a heart rate variable (HRV), i.e. the variation in time interval between consecutive heartbeats, of the user of earphones 100. For example, processor 165 may calculate the HRV using the data collected by sensor 122 based on a time domain methods, frequency domain methods, and other methods known in the art that calculate HRV based on data such as the mean heart rate, the change in pulse rate over a time interval, and other data used in the art to estimate HRV.

In further embodiments, logic circuits of processor 165 may further detect, calculate, and store metrics such as the amount of physical activity, sleep, or rest over a period of time, or the amount of time without physical activity over a period of time. The logic circuits may use the HRV, the metrics, or some combination thereof to calculate a recovery score. In various embodiments, the recovery score may indicate the user's physical condition and aptitude for further physical activity for the current day. For example, the logic circuits may detect the amount of physical activity and the amount of sleep a user experienced over the last 48 hours, combine those metrics with the user's HRV, and calculate a recovery score. In various embodiments, the calculated recovery score may be based on any scale or range, such as, for example, a range between 1 and 10, a range between 1 and 100, or a range between 0% and 100%.

During audio playback, earphones 100 wirelessly receive audio data using wireless transceiver 180. The audio data is processed by logic circuits of audio processor 160 into electrical signals that are delivered to respective drivers 113 and 123 of speaker 114 and speaker 124 of earphones 110 and 120. The electrical signals are then converted to sound using the drivers. Any driver technologies known in the art or later developed may be used. For example, moving coil drivers, electrostatic drivers, electret drivers, orthodynamic drivers, and other transducer technologies may be used to generate playback sound.

The wireless transceiver 180 is configured to communicate biometric and audio data using available wireless communications standards. For example, in some embodiments, the wireless transceiver 180 may be a BLUETOOTH transmitter, a ZIGBEE transmitter, a Wi-Fi transmitter, a GPS transmitter, a cellular transmitter, or some combination thereof. Although FIG. 2B illustrates a single wireless transceiver 180 for both transmitting biometric data and receiving audio data, in an alternative embodiment, a transmitter dedicated to transmitting only biometric data to a computing device may be used. In this alternative embodiment, the transmitter may be a low energy transmitter such as a near field communications (NFC) transmitter or a BLUETOOTH low energy (LE) transmitter. In implementations of this particular embodiment, a separate wireless receiver may be provided for receiving high fidelity audio data from an audio source. In yet additional embodiments, a wired interface (e.g., micro-USB) may be used for communicating data stored in memories 165 and 175.

FIG. 2B also shows that the electrical components of headphones 100 are powered by a battery 190 coupled to power circuity 191. Any suitable battery or power supply technologies known in the art or later developed may be used. For example, a lithium-ion battery, aluminum-ion battery, piezo or vibration energy harvesters, photovoltaic cells, or other like devices can be used. In embodiments, battery 190 may be enclosed in earphone 110 or earphone 120. Alternatively, battery 102 may be enclosed in controller 130. In embodiments, the circuitry may be configured to enter a low-power or inactive mode when earphones 100 are not in use. For example, mechanisms such as, for example, an on/off switch, a BLUETOOTH transmission disabling button, or the like may be provided on controller 130 such that a user may manually control the on/off state of power-consuming components of earphones 100.

It should be noted that in various embodiments, processors 160 and 165, memories 170 and 175, wireless transceiver 180, motion sensor 121, optical heartrate sensor 122, and battery 190 may be enclosed in and distributed throughout any one or more of earphone 110, earphone 120, and controller 130. For example, in one particular embodiment, processor 165 and memory 175 may be enclosed in earphone 120 along with optical heartrate sensor 122 and motion sensor 121. In this particular embodiment, these four components are electrically coupled to the same printed circuit board (PCB) enclosed in earphone 120. It should also be noted that although audio processor 160 and biometric processor 165 are illustrated in this exemplary embodiment as separate processors, in an alternative embodiment the functions of the two processors may be integrated into a single processor.

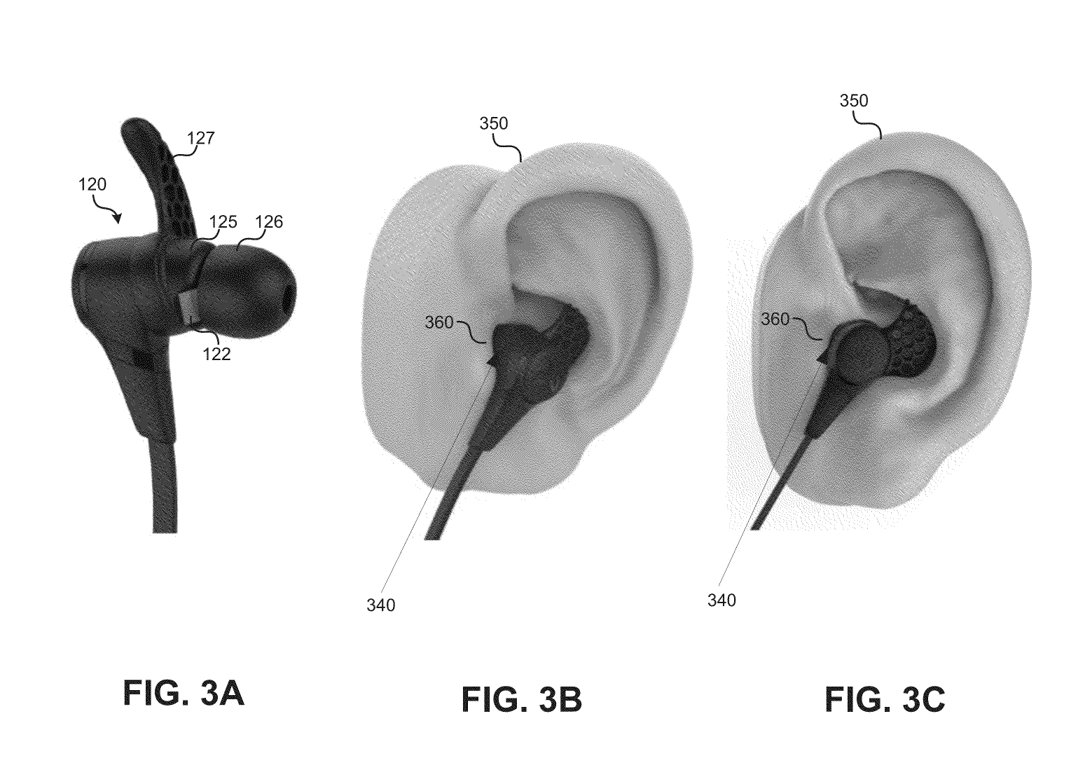

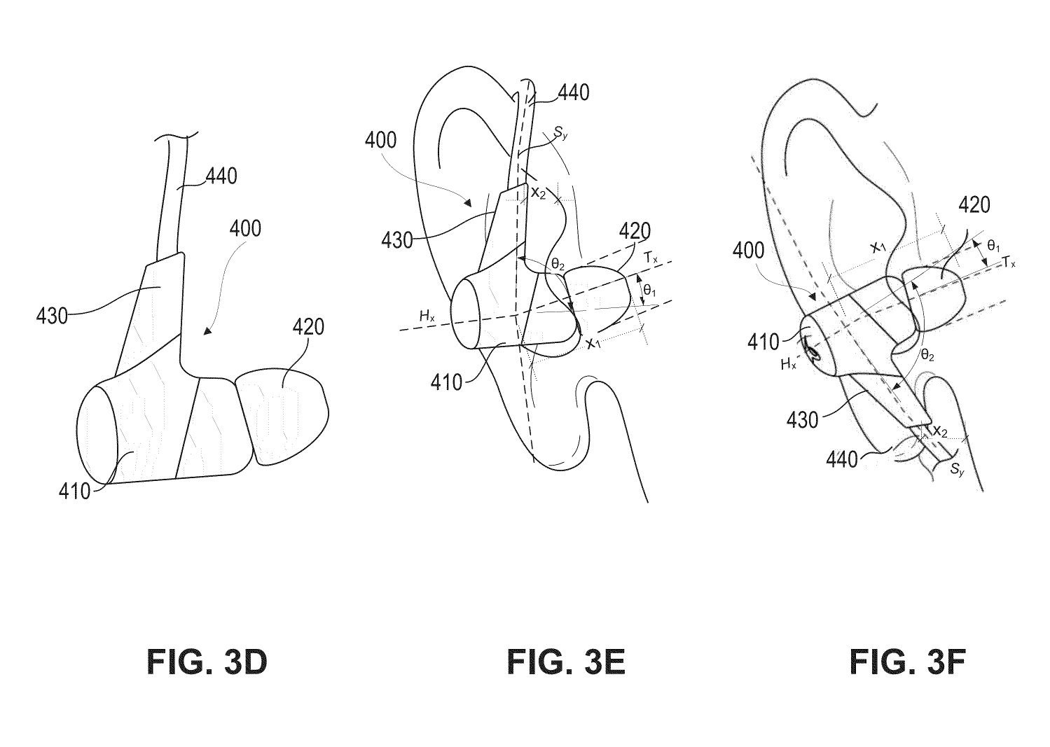

FIG. 3A illustrates a perspective view of one embodiment of an earphone 120, including an optical heartrate sensor 122, in accordance with the technology disclosed herein. FIG. 3A will be described in conjunction with FIGS. 3B-3C, which are perspective views illustrating placement of heartrate sensor 122 when earphone 120 is worn in a user's ear 350. It is important to note here that the earphone depicted in FIG. 3A is configured to be placed in a right ear of a human user, and that the earphones depicted in FIGS. 3B-3C depict the earphones being worn in a user's left ear. These alternative views are included to demonstrate that the features disclosed herein with respect to earphone 120 may be implemented in a left earphone, a right earphone, a single earphone, or both earphones. Indeed, the functionality of earphone 120 as disclosed herein, may in some embodiments be implemented in earphone 110 alone, or in combination with the same functionality implemented in earphone 120. Moreover, in some embodiments, ear cushion 127 may be removable and invertably reattached to earphone 120 such that earphone 120 may be worn in a user's left ear rather than a user's right ear. Accordingly, though the earphones in FIGS. 3B-3C will be referred to as earphone 120, the technology disclosed herein is operable whether earphone 120 is utilized as a right earphone or a left earphone.

As illustrated in FIG. 3A, earphone 120 includes a body 125, tip 126, ear cushion 127, and an optical heartrate sensor 122. Optical heartrate sensor 122 protrudes from a frontal side of body 125, proximal to tip 126 and where the earphone's nozzle (not shown) is present. FIGS. 3B-3C illustrate the optical sensor and ear interface 340 when an such as earphone 120 is worn in a user's ear 350. When an earphone such as earphone 120 is worn in the ear 350 of a user, optical heartrate sensor 122 is proximal to the interior side of a user's tragus 360.

In this embodiment, optical heartrate sensor 122 illuminates the skin of the interior side of the ear's tragus 360 with a light-emitting diode (LED). The light penetrates through the epidermal layers of the skin to underlying blood vessels. A portion of the light is absorbed and a portion is reflected back. The light reflected back through the skin is then obtained with a receiver (e.g., a photodiode) of optical heartrate sensor 122 and used to determine changes in the user's blood flow, thereby permitting measurement of the user's heart rate and HRV.

In various embodiments, earphones 100 may be dual-fit earphones shaped to comfortably and securely be worn in either an over-the-ear configuration or an under-the-ear configuration. The secure fit provided by such embodiments keeps the optical heartrate sensor 122 in place on the interior side of the ear's tragus 360, thereby ensuring accurate and consistent measurements of a user's heartrate.

FIGS. 3D and 3E are cross-sectional views illustrating one such embodiment of dual-fit earphones 400 being worn in an over-the-ear configuration. FIG. 3F illustrates dual-fit earphones 400 in an under-the-ear configuration.

As illustrated, earphone 400 includes housing 410, tip 420, strain relief 430, and cord or cable 440. The proximal end of tip 420 mechanically couples to the distal end of housing 410. Similarly, the distal end of strain relief 430 mechanically couples to a side (e.g., the top side) of housing 410. Furthermore, the distal end of cord 440 is disposed within and secured by the proximal end of strain relief 430. The longitudinal axis of the housing, Hₓ, forms angle θ₁ with respect to the longitudinal axis of the tip, Tₓ. The longitudinal axis of the strain relief, S_{y}, aligns with the proximal end of strain relief 430 and forms angle θ₂ with respect to the axis Hₓ. In several embodiments, θ₁ is greater than 0 degrees (e.g., Tₓ extends in a non-straight angle from Hₓ, or in other words, the tip 420 is angled with respect to the housing 410). In some embodiments, θ₁ is selected to approximate the ear canal angle of the wearer. For example, θ₁ may range between 5 degrees and 15 degrees. Also in several embodiments, θ₂ is less than 90 degrees (e.g., S_{y} extends in a non-orthogonal angle from Hₓ, or in other words, the strain relief 430 is angled with respect to a perpendicular orientation with housing 410). In some embodiments, θ₂ may be selected to direct the distal end of cord 440 closer to the wearer's ear. For example, θ₂ may range between 75 degrees and 85 degrees

As illustrated, x₁ represents the distance between the distal end of tip 420 and the intersection of strain relief longitudinal axis S_{y} and housing longitudinal axis Hₓ. One of skill in the art would appreciate that the dimension x₁ may be selected based on several parameters, including the desired fit to a wearer's ear based on the average human ear anatomical dimensions, the types and dimensions of electronic components (e.g., optical sensor, motion sensor, processor, memory, etc.) that must be disposed within the housing and the tip, and the specific placement of the optical sensor. In some examples, x₁ may be at least 18 mm. However, in other examples, x₁ may be smaller or greater based on the parameters discussed above.

Similarly, as illustrated, x₂ represents the distance between the proximal end of strain relief 430 and the surface wearer's ear. In the configuration illustrated, θ₂ may be selected to reduce x₂, as well as to direct the cord 440 towards the wearer's ear, such that cord 440 may rest in the crevice formed where the top of the wearer's ear meets the side of the wearer's head. In some embodiments, θ₂ may range between 75 degrees and 85 degrees. In some examples, strain relief 430 may be made of a flexible material such as rubber, silicone, or soft plastic such that it may be further bent towards the wearer's ear. Similarly, strain relief 430 may comprise a shape memory material such that it may be bent inward and retain the shape. In some examples, strain relief 430 may be shaped to curve inward towards the wearer's ear.

In some embodiments, the proximal end of tip 420 may flexibly couple to the distal end of housing 410, enabling a wearer to adjust θ₁ to most closely accommodate the fit of tip 420 into the wearer's ear canal (e.g., by closely matching the ear canal angle).

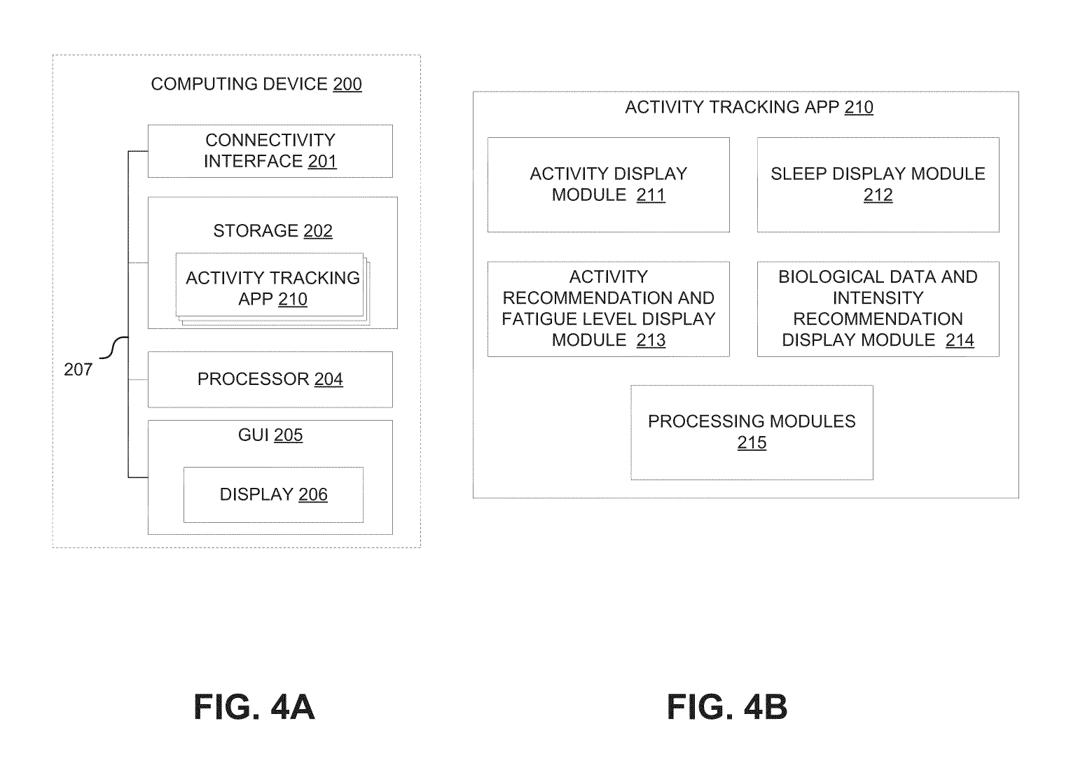

As one having skill in the art would appreciate from the above description, earphones 100 in various embodiments may gather biometric user data that may be used to track a user's activities and activity level. That data may then be made available to a computing device, which may provide a GUI for interacting with the data using a software activity tracking application installed on the computing device. FIG. 4A is a block diagram illustrating example components of one such computing device 200 including an installed activity tracking application 210.

As illustrated in this example, computing device 200 comprises a connectivity interface 201, storage 202 with activity tracking application 210, processor 204, a graphical user interface (GUI) 205 including display 206, and a bus 207 for transferring data between the various components of computing device 200.

Connectivity interface 201 connects computing device 200 to earphones 100 through a communication medium. The medium may comprise a wireless network system such as a BLUETOOTH system, a ZIGBEE system, an Infrared (IR) system, a Radio Frequency (RF) system, a cellular network, a satellite network, a wireless local area network, or the like. The medium may additionally comprise a wired component such as a USB system.

Storage 202 may comprise volatile memory (e.g. RAM), non-volatile memory (e.g. flash storage), or some combination thereof. In various embodiments, storage 202 may store biometric data collected by earphones 100. Additionally, storage 202 stores an activity tracking application 210, that when executed by processor 204, allows a user to interact with the collected biometric information.

In various embodiments, a user may interact with activity tracking application 210 via a GUI 205 including a display 206, such as, for example, a touchscreen display that accepts various hand gestures as inputs. In accordance with various embodiments, activity tracking application 210 may process the biometric information collected by earphones 100 and present it via display 206 of GUI 205. Before describing activity tracking application 210 in further detail, it is worth noting that in some embodiments earphones 100 may filter the collected biometric information prior to transmitting the biometric information to computing device 200. Accordingly, although the embodiments disclosed herein are described with reference to activity tracking application 210 processing the received biometric information, in various implementations various preprocessing operations may be performed by a processor 160, 165 of earphones 100.

In various embodiments, activity tracking application 210 may be initially configured/setup (e.g., after installation on a smartphone) based on a user's self-reported biological information, sleep information, and activity preference information. For example, during setup a user may be prompted via display 206 for biological information such as the user's gender, height, age, and weight. Further, during setup the user may be prompted for sleep information such as the amount of sleep needed by the user and the user's regular bed time. Further, still, the user may be prompted during setup for a preferred activity level and activities the user desires to be tracked (e.g., running, walking, swimming, biking, etc.) In various embodiments, described below, this self-reported information may be used in tandem with the information collected by earphones 100 to display activity monitoring information using various modules.

Following setup, activity tracking application 210 may be used by a user to monitor and define how active the user wants to be on a day-to-day basis based on the biometric information (e.g., accelerometer information, optical heart rate sensor information, etc.) collected by earphones 100. As illustrated in FIG. 4B, activity tracking application 210 may comprise various display modules, including an activity display module 211, a sleep display module 212, an activity recommendation and fatigue level display module 213, and a biological data and intensity recommendation display module 214. Additionally, activity tracking application 210 may comprise various processing modules 215 for processing the activity monitoring information (e.g., optical heartrate information, accelerometer information, gyroscope information, etc.) collected by the earphones or the biological information entered by the users. These modules may be implemented separately or in combination. For example, in some embodiments activity processing modules 215 may be directly integrated with one or more of display modules 211-214.

As will be further described below, each of display modules 211-214 may be associated with a unique display provided by activity tracking app 210 via display 206. That is, in some embodiments, activity display module 211 may have an associated activity display, sleep display module 212 may have an associated sleep display, activity recommendation and fatigue level display module 213 may have an associated activity recommendation and fatigue level display, and biological data and intensity recommendation display module 214 may have an associated biological data and intensity recommendation display.

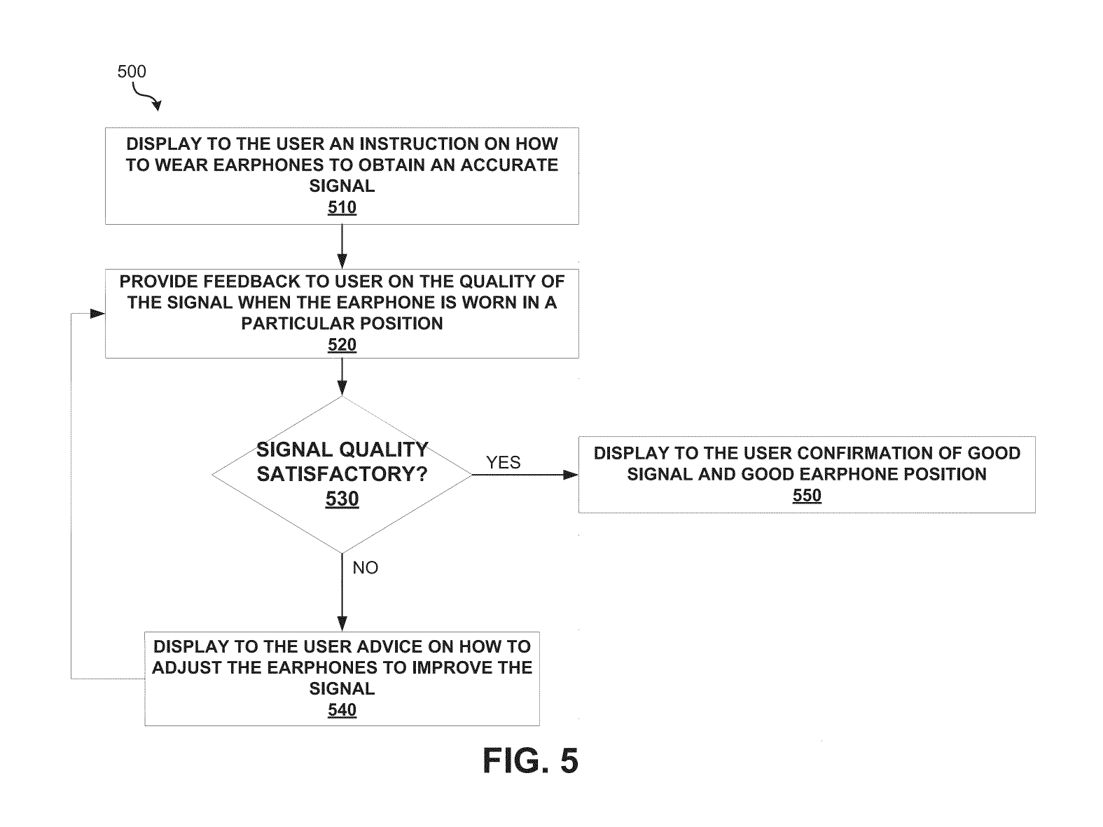

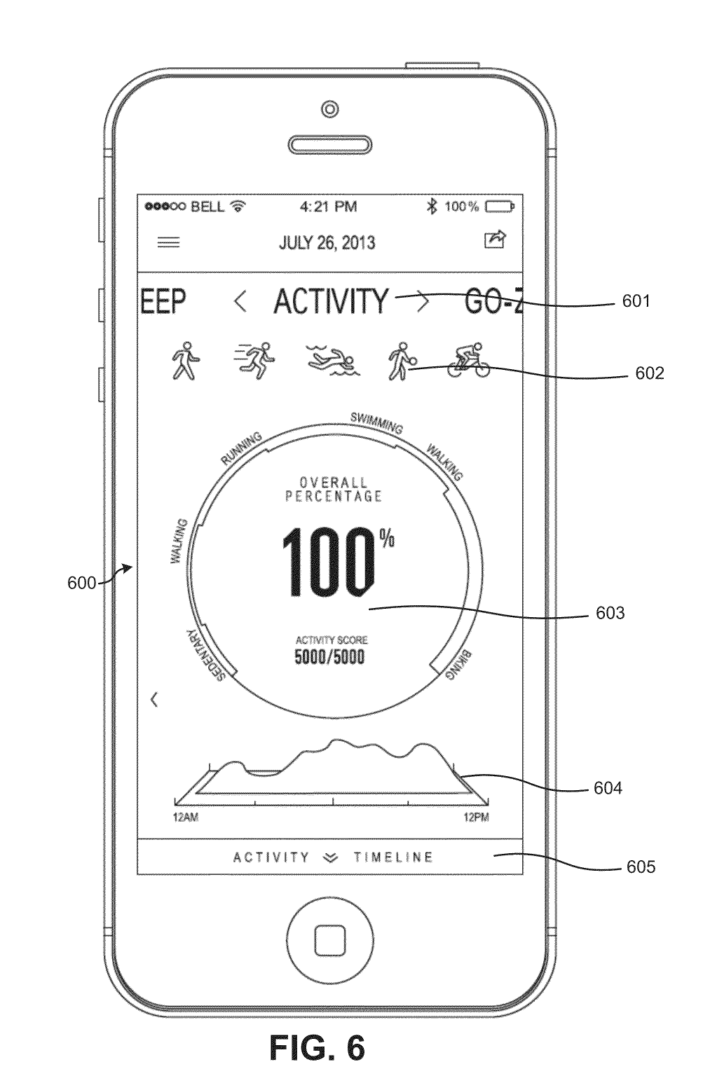

In embodiments, application 210 may be used to display to the user an instruction for wearing and/or adjusting earphones 100 if it is determined that optical heartrate sensor 122 and/or motion sensor 121 are not accurately gathering motion data and heart rate data. FIG. 5 is an operational flow diagram illustrating one such method 500 of an earphone adjustment feedback loop with a user that ensures accurate biometric data collection by earphones 100. At operation 510, execution of application 210 may cause display 206 to display an instruction to the user on how to wear earphones 100 to obtain an accurate and reliable signal from the biometric sensors. In embodiments, operation 510 may occur once after installing application 210, once a day (e.g., when user first wears the earphones 100 for the day), or at any custom and/or predetermined interval.

At operation 520, feedback is displayed to the user regarding the quality of the signal received from the biometric sensors based on the particular position that earphones 100 are being worn. For example, display 206 may display a signal quality bar or other graphical element. At decision 530, it is determined if the biosensor signal quality is satisfactory for biometric data gathering and use of application 210. In various embodiments, this determination may be based on factors such as, for example, the frequency with which optical heartrate sensor 122 is collecting heart rate data, the variance in the measurements of optical heartrate sensor 122, dropouts in heart rate measurements by sensor 122, the signal-to-noise ratio approximation of optical heartrate sensor 122, the amplitude of the signals generated by the sensors, and the like.

If the signal quality is unsatisfactory, at operation 540, application 210 may cause display 206 to display to the user advice on how to adjust the earphones to improve the signal, and operations 520 and decision 530 may subsequently be repeated. For example, advice on adjusting the strain relief of the earphones may be displayed. Otherwise, if the signal quality is satisfactory, at operation 550, application may cause display 206 to display to the user confirmation of good signal quality and/or good earphone position. Subsequently, application 210 may proceed with normal operation (e.g., display modules 211-214). FIGS. 6,12-14 illustrate a particular exemplary implementation of a GUI for app 210 comprising displays associated with each of display modules 211-214.

FIG. 6 illustrates an activity display 600 that may be associated with an activity display module 211. In various embodiments, activity display 600 may visually present to a user a record of the user's activity. As illustrated, activity display 600 may comprise a display navigation area 601, activity icons 602, activity goal section 603, live activity chart 604, and activity timeline 605. As illustrated in this particular embodiment, display navigation area 601 allows a user to navigate between the various displays associated with modules 211-214 by selecting “right” and “left” arrows depicted at the top of the display on either side of the display screen title. An identification of the selected display may be displayed at the center of the navigation area 601. Other selectable displays may displayed on the left and right sides of navigation area 601. For example, in this embodiment the activity display 600 includes the identification “ACTIVITY” at the center of the navigation area. If the user wishes to navigate to a sleep display in this embodiment, the user may select the left arrow. In implementations where device 200 includes a touch screen display, navigation between the displays may be accomplished via finger swiping gestures. For example, in one embodiment a user may swipe the screen right or left to navigate to a different display screen. In another embodiment, a user may press the left or right arrows to navigate between the various display screens.

In various embodiments, activity icons 602 may be displayed on activity display 600 based on the user's predicted or self-reported activity. For example, in this particular embodiment activity icons 602 are displayed for the activities of walking, running, swimming, sport, and biking, indicating that the user has performed these five activities. In one particular embodiment, one or more modules of application 210 may estimate the activity being performed (e.g., sleeping, walking, running, or swimming) by comparing the data collected by a biometric earphone's sensors to pre-loaded or learned activity profiles. For example, accelerometer data, gyroscope data, heartrate data, or some combination thereof may be compared to preloaded activity profiles of what the data should look like for a generic user that is running, walking, or swimming. In implementations of this embodiment, the preloaded activity profiles for each particular activity (e.g., sleeping, running, walking, or swimming) may be adjusted over time based on a history of the user's activity, thereby improving the activity predictive capability of the system. In additional implementations, activity display 600 allows a user to manually select the activity being performed (e.g., via touch gestures), thereby enabling the system to accurately adjust an activity profile associated with the user-selected activity. In this way, the system's activity estimating capabilities will improve over time as the system learns how particular activity profiles match an individual user. Particular methods of implementing this activity estimation and activity profile learning capability are described in U.S. patent application Ser. No. 14/568,835, filed Dec. 12, 2014, titled “System and Method for Creating a Dynamic Activity Profile”, and which is incorporated herein by reference in its entirety.

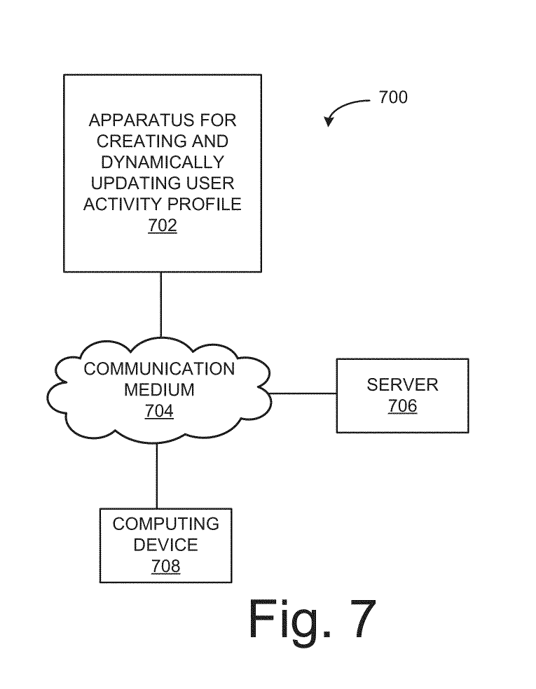

For example, FIG. 7 is a schematic block diagram illustrating an example implementation of system 700 for creating a dynamic user activity profile in in connection with the activity profile learning capability described above with reference to FIG. 6. System 700 includes apparatus 702 for creating and dynamically updating a user activity profile, communication medium 704, server 706, and computing device 708. Embodiments of system 700 are capable of capturing and tracking robust information related to a user's activity, including information about the user's activity type, duration, intensity, and so on. Moreover, embodiments of system 700 create and dynamically update a user activity profile based on the captured and tracked user activity information. This dynamically updated user activity profile allows system 700, in various embodiments, to provide user-specific recommendations regarding activity, including target goals and the like. Being user-specific and dynamic, the recommendations provided by system 700 may be personalized, so as to push the user to progress, while also being based on the user's actual activity, including up-to-date trends and statistics related thereto, thus being realistically achievable by the user. Such personalized, yet realistic goals may be more effective in terms of driving the user's progress than, for example, overly aggressive goals that may be discouraging or may result in injury, or goals that are not sufficiently aggressive to push the user's limits.

In some embodiments, communication medium 704 may be used to connect or communicatively couple apparatus 702, server 706, and/or computing device 708 to one another or to a network, and communication medium 704 may be implemented in a variety of forms. For example, communication medium 704 may include an Internet connection, such as a local area network (“LAN”), a wide area network (“WAN”), a fiber optic network, internet over power lines, a hard-wired connection (e.g., a bus), and the like, or any other kind of network connection. Communication medium 704 may be implemented using any combination of routers, cables, modems, switches, fiber optics, wires, radio (e.g., microwave/RF links), and the like. Communication medium 704 may be implemented using various wireless standards, such as Bluetooth®, Wi-Fi, 3GPP standards (e.g., 4G LTE), etc. Upon reading the present disclosure, one of skill in the art will recognize other ways to implement communication medium 704 to establish, for example, a communication link 300 as illustrated in FIG. 1, for communications purposes.

Server 706 directs communications made over communication medium 704. Server 706 may include, for example, an Internet server, a router, a desktop or laptop computer, a smartphone, a tablet, a processor, a module, or the like, and may be implemented in various forms, include, for example, an integrated circuit, a printed circuit board, or in a discrete housing/package. In one embodiment, server 706 directs communications between communication medium 704 and computing device 708. For example, server 706 may update information stored on computing device 708, or server 706 may send/receive information to/from computing device 708 in real time.

Computing device 708 may take a variety of forms, such as a desktop or laptop computer, a smartphone, a tablet, a smartwatch or other wearable electronic device, a processor, a module, or the like. In some embodiments, computing device 708 is embodied in computing device 200 depicted in FIG. 4A. In other embodiments, computing device 708 may be embodied in earphones 100 of FIGS. 1-2. By way of illustration, computing device 708 may be a processor or module embedded in a wearable sensor (e.g. biometric earphones 100), a bracelet, a smart-watch, a piece of clothing, an accessory, and so on. Computing device 708 may also be, for example, substantially similar to devices embedded biometric earphones 100, as illustrated in FIGS. 1-3F and described hereinabove. Computing device 708 may communicate with other devices over communication medium 704 with or without the use of server 706. In one embodiment, computing device 708 includes apparatus 702 for creating and dynamically updating a user activity profile. In various embodiments, apparatus 702 may be used to perform various processes described herein and/or may be used to execute various operations described herein with regard to one or more disclosed systems and methods. For example, in some embodiments, apparatus 702 includes a pair of biometric earphones 100. Upon studying the present disclosure, one of skill in the art will appreciate that system 700 may include multiple apparatus 702, servers 706, and/or computing devices 708, and that apparatus 702 may be embodied in or part of computing device 708, and likewise that a computing device 708 may be embodied in an apparatus 702.

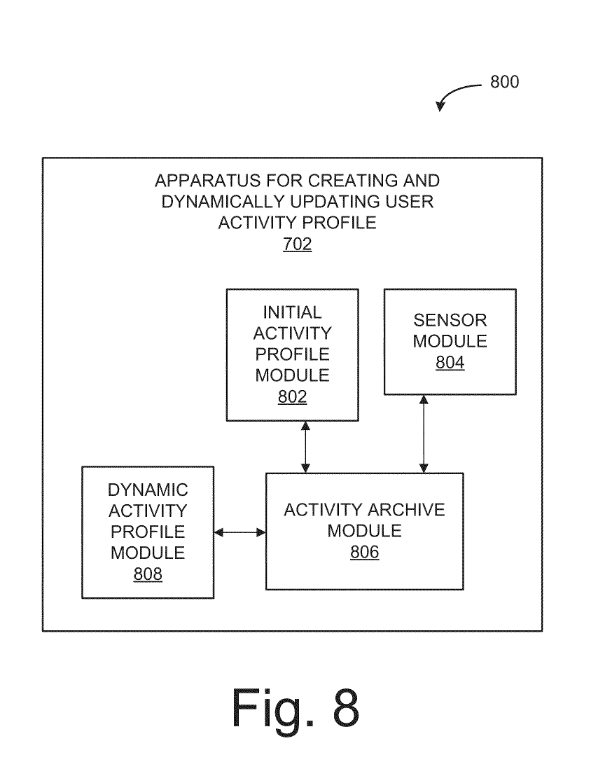

FIG. 8 is a schematic block diagram illustrating an embodiment of apparatus 800 for creating a dynamic user activity profile. As illustrated, apparatus 800 includes apparatus 702, which, in turn, includes initial activity profile module 802, sensor module 804, activity archive module 806, and dynamic activity profile module 808. In various implementations of the disclosure, apparatus 702 creates a dynamic user activity profile as follows: initial activity profile module 802 creates an activity profile for a user; sensor module 804 monitors or tracks the user's activity to generate activity information; the activity information may be fed to activity archive module 806; activity archive module 806 maintains an activity archive that includes the activity information; and dynamic activity profile module 808 updates the activity profile based on the activity archive. In this manner, the dynamic activity profile module 808, in conjunction with the other, above-mentioned modules of apparatus 702, may create/update the user activity profile to be a dynamic profile that captures up-to-date information and trends regarding the user's activity. Additional aspects and features of initial activity profile module 802, sensor module 804, activity archive module 806, and dynamic activity profile module 808, are described below in further detail with regard to various processes and/or methods disclosed herein.

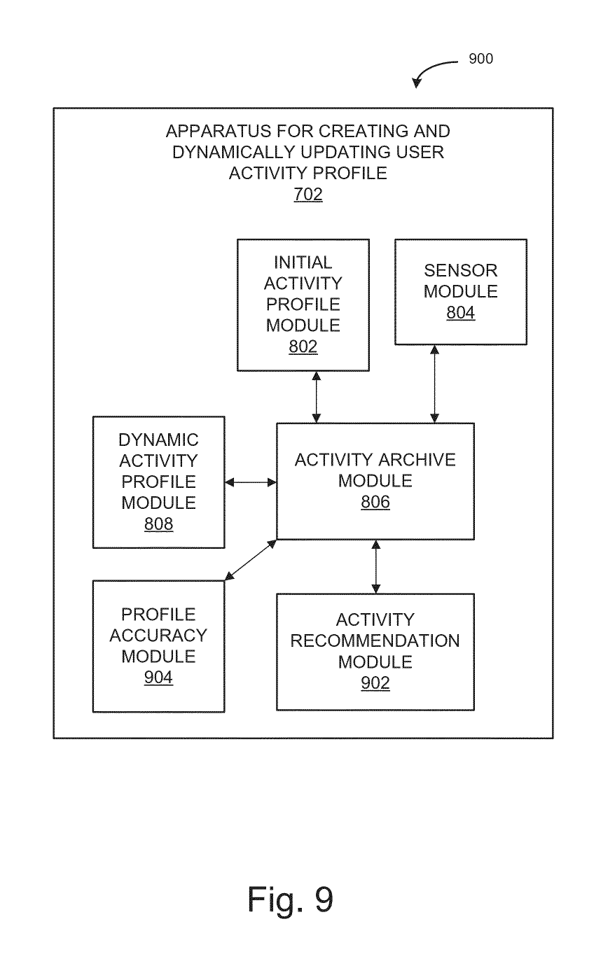

FIG. 9 is a schematic block diagram illustrating an embodiment of apparatus 900 for creating a dynamic user activity profile. As shown in FIG. 9, apparatus 900 includes apparatus 702, which, in turn, includes initial activity profile module 802, sensor module 804, activity archive module 806, and dynamic activity profile module 808. Apparatus 900 also includes activity recommendation module 902 and profile accuracy module 904. Activity recommendation module 902 provides a recommendation related to the user's activity. The recommendation is based on the activity profile that is created by initial activity profile module 802 and updated by dynamic activity profile module 808. Having dynamic input regarding the activity profile (e.g., by way of archive module 806), activity recommendation module 902 may provide a user-specific, up-to-date activity recommendation that is both personalized and realistic for the user. Profile accuracy module 904 provides an indication of an estimated level of accuracy of the activity profile created by initial activity profile module 802 and updated by dynamic activity profile module 808. The estimated level of accuracy is based on the amount and consistency of activity information in the activity archive maintained by activity archive module 806. Additional aspects and features of activity recommendation module 902 and profile accuracy module 904 are described below in further detail with regard to various processes and/or methods disclosed herein.

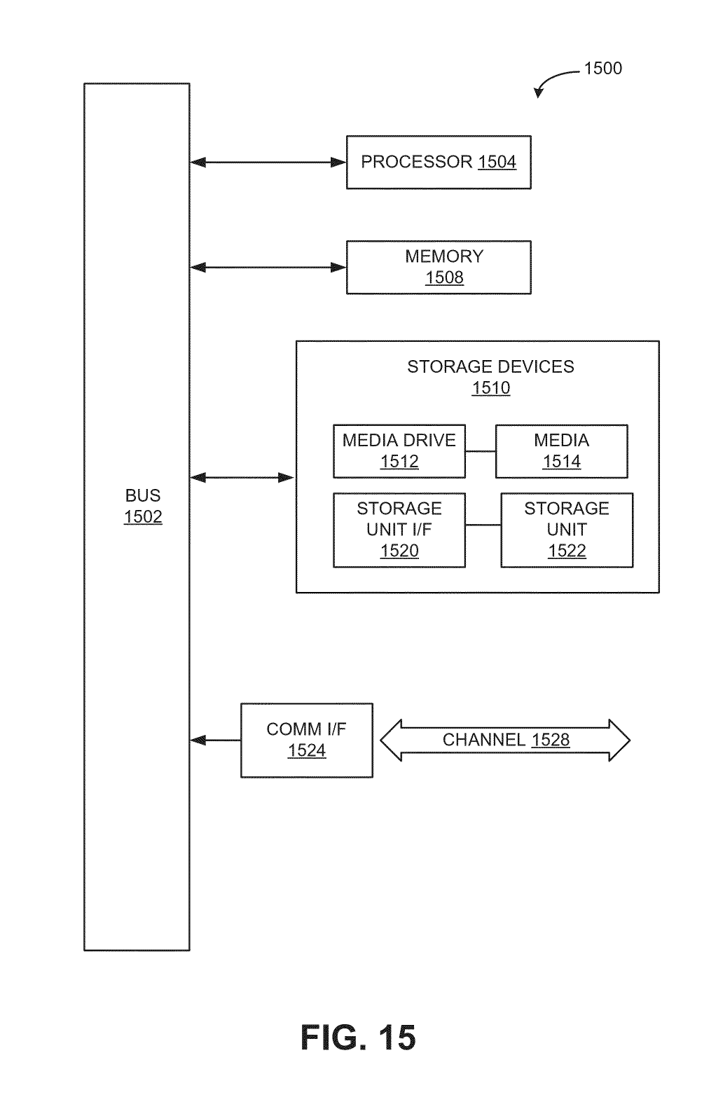

In various embodiments of the disclosure, one or more of initial activity profile module 802, sensor module 804, activity archive module 806, dynamic activity profile module 808, activity recommendation module 902, and profile accuracy module 904, is embodied in a wearable device, such as, for example, biometric earphones 100. Moreover, any of the modules described herein may be embodied in one or more biometric earphones 100, other wearable devices, or other hardware/devices (e.g., mobile devices), as will be appreciated by one of skill in the art after reading the present disclosure. Furthermore, any of the modules described herein may connect and/or communicatively couple to other modules described herein via communication medium 704. Example structures of these modules will be described in further detail herein below with regard to FIG. 15.

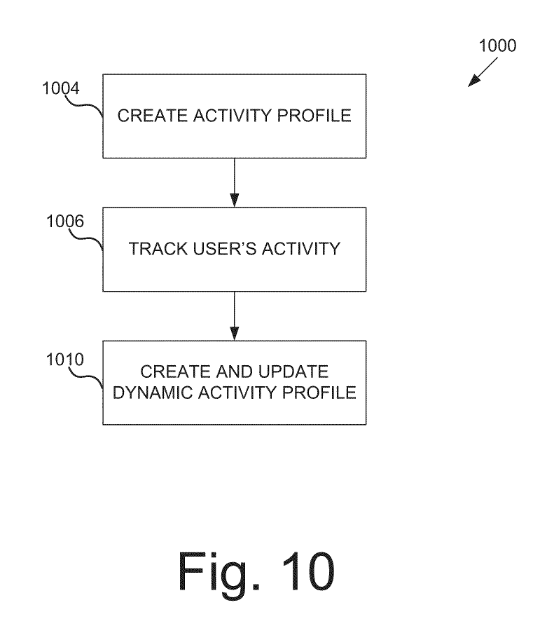

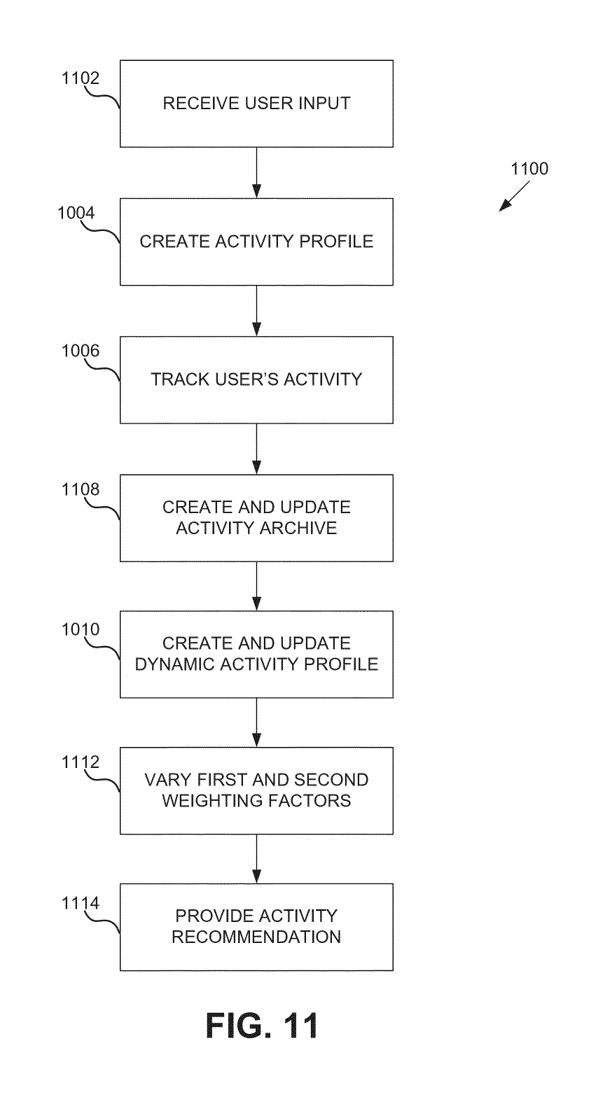

FIGS. 10 and 11 contain operational flow diagrams illustrating example embodiments of methods 1000 and 1100, respectively, for creating a dynamic user activity profile, in accordance with the present disclosure. The operations of methods 1000 and 1100 create and update a dynamic activity profile that is based on robust tracking of user activity and that may take into account and analyze up-to-date trends and patterns related to user activity. For example, the operations of methods 1000 and 1100 may account for patterns of activity and recovery to simulate learning the capabilities and tendencies of a user. As such, methods 1000 and/or 1100 may include providing recommendations of goals for activity levels/types that are highly tailored to the user's specific characteristics, and that are personalized, yet realistic for the user. In example implementations of methods 1000 and 1100, apparatus 702, earphones 100, and/or one or more subcomponents/modules thereof, perform various operations of methods 1000 and/or 1100, which operations are described in further detail below.

Referring to FIGS. 10 and 11, at operation 1004, methods 1000 and 1100 involve creating an activity profile for a user. Operation 1004 may be performed as an initial matter, for example, before any user activity is tracked and before a dynamic user activity profile is created (e.g., according to operations described subsequently). By way of illustration, one embodiment of method 1100 entails receiving user input to an activity questionnaire—e.g., at operation 1102, depicted FIG. 11. In such an embodiment, the activity profile created at operation 1004 is based on the user input to the activity questionnaire. Additionally, the user input may be solicited by other means (e.g. GUI 205, display 600, etc.) that will become apparent to one of skill in the art upon reading the present disclosure.

The activity questionnaire may be designed to facilitate the user and/or prompt user input information regarding the user, with specific regard to information about the user's activities and lifestyle tendencies. This may entail the user inputting information about the user's physical profile (e.g., height, weight, age, gender, etc.), information about the user's sleep habits (e.g., average number of hours per night, and the like), information about the user's activity levels and/or lifestyle (e.g., very active, moderately active, sedentary, etc.), and information about the user's activity aspirations (e.g., train for a race, lose weight, maintain current condition, and so on).

The activity questionnaire may be implemented, in some embodiments, using a graphical user interface (e.g. GUI 205, display 600, etc.), drop-down menus, and so one—for example, on a computing device, smartphone, or wearable electronic device. The user may also select various preferred or actual activity types, intensities, durations, and may input additional information, for example, information regarding past/current injuries, the user's schedule, training partners, music/media preferences for workouts or other activities, and the like. This type of user information may aid in tailoring the types of activities recommended for the user in accordance with various embodiments of methods 1000 and 1100. In some instances, operation 1002 may be performed by a module and/or computing device that is separate from but connected to apparatus 702. By way of illustration, the questionnaire and related GUI may be presented to the user on a smartphone display screen, while apparatus 702 may reside in a wearable device (e.g., biometric earphones 100).

In various embodiments of methods 1000 and 1100, creating the activity profile (operation 1004) includes modifying the user input according to normative statistical data. The modification may depend on the type of user input received, but generally is used to determine a range of activity attributes that are typical for individuals similar to the user. For example, if the user input includes the user's age, but does not include information on the user's typical or desired activity level, the normative statistical data may be used to create an activity profile that reflects the typical activity level for a person of the user's age. The normative statistical data may be based on averages and/or other weighted data collected from statistically significant groups of individuals, and may, in some instances, be based on publicly available information. Other illustrative examples of normative statistical data that may be used to modify the user input include age-related data, physiological data, sleep data, and the like. Being based on normative data in combination with varying amounts of user-input data (e.g., ranging from minimal to extensive based on the how the user decides to populate the questionnaire), the activity profile as initially created may be somewhat tailored to the user, though not based on actually tracked/measured user activity information. As such, the modified user input may provide a useful baseline upon which the dynamic activity profile may be built, according to subsequent operations of methods 1000 and 1100. Operation 1004, in various example implementations, is performed by initial activity profile module 802.

At operation 1006, methods 1000 and 1100 involve tracking the user's activity. Tracking the user's activity may entail monitoring the user's movement (e.g., using a wearable device), to determine, for example, an activity type, intensity, duration, and so on. In some cases, operation 1006 is accomplished using a sensor configured to be attached to a user's body (e.g., by way of a wearable device such as biometric earphones 100). Such a sensor may include a gyroscope or accelerometer to detect movement, and a heart-rate sensor, each of which may be embedded in one or more of a computing device 708 (e.g. computing device 200) that can be worn on the arm, wrist, or leg of a user, and/or a pair of biometric earphones 100 that a user can wear in the user's ear(s). Various embodiments of operation 1006 may entail using sensor module 804.

By way of illustration, the activity type may be selected from typical activities, such as running, walking, sleeping, swimming, bicycling, skiing, surfing, resting, working, and so on. The activity intensity may be represented on a numeric scale. For example, the activity intensity may include a number ranging from one to ten (representing increasing activity intensity). And the activity intensity may be associated with the vigorousness of an activity. In other embodiments, the activity intensity may be represented by ranges of heart rates and/or breathing rates, which may also be determined as part of tracking the user's activity.

In this regard, tracking the user's activity includes, in some instances of the disclosed methods 1000 and 1100 for creating a dynamic user profile, determining an activity level of the user, which may further entail capturing and/or compiling data regarding the user's fatigue/recovery levels (e.g., as described above in reference to FIGS. 1-4B). Moreover, in additional embodiments, tracking the user's activity includes tracking a set of activity parameters. The set of activity parameters may include, for example, HRV (and/or fatigue/recovery level), sleep duration, sleep quality, subjective feedback from the user (e.g., how the user feels after a certain activity), previous user activity levels, and training load data/models). To expound, training load models may be loaded to provide comparison bases for the user's activity level, as tracked according to operation 1006, for example. Training load models may include ideal or otherwise previously determined progress/training milestones that a user may be advised to accomplish to achieve an end-goal, such as to be ready for a race, or the like.

As described, fatigue level is one example of an activity parameter that may be tracked as part of tracking the user's activity level at operation 1006. The fatigue level may be a function of recovery and/or may be described in terms of recovery (e.g., as described above in reference to FIGS. 1-6). Further, the fatigue level may be detected using various techniques. For example, the fatigue level may be detected by measuring the user's HRV (e.g., using optical heartrate sensor, logic circuits and processors, at detailed above in reference to FIG. 1-5). When HRV is more consistent (i.e., steady, consistent amount of time between heartbeats), the fatigue level may be higher. In other words, the body may be less fresh and not well rested. By contrast, when HRV is more sporadic (i.e., amount of time between heartbeats varies largely), the fatigue level may be lower. In various embodiments, the fatigue level is described in terms of an HRV or HRV score.

In various implementations, at operation 1006, HRV may be measured/determined in a number of ways (also discussed above in reference to FIG. 1-5). In such embodiments, optical heartrate sensor 122 may be used to estimate a heart rate variable (HRV), i.e. the variation in time interval between consecutive heartbeats, of the user of earphones 100. For example, processor 165 may calculate the HRV using the data collected by sensor 122 based on a time domain methods, frequency domain methods, and other methods known in the art that calculate HRV based on data such as the mean heart rate, the change in pulse rate over a time interval, and other data used in the art to estimate HRV.

In still further embodiments, HRV is determined using the combination of optical heartrate sensor 122 and one or more finger biosensors coupled to earphones 100, or embodied within another computing device (e.g. computing device 200, apparatus 702, etc.). This combination can allow the sensors, which in one embodiment may be conductive, to measure an electrical potential through the body. Information about the electrical potential provides cardiac information (e.g., HRV, fatigue level, heart rate information, and so on) and such information may be processed, analyzed, and/or tracked in accordance with tracking the user's activity level in methods 1000 and 1100. In other instances, HRV is determined using sensors that monitor other parts of the user's body, rather than the tragus and/or finger. For example, the sensor may monitor the ankle, leg, arm, and/or torso.

Fatigue level may be detected, in some cases, based solely on the HRV measured. Additionally, fatigue level may be based at least partially on other measurements or aspects of the user's activity level that may be tracked at operation 1006. For example, fatigue level may be based on the amount of sleep that is measured for the previous night, the duration and/or type of user activity, and the intensity of the activity determined for a previous time period (e.g., exercise activity level in the last twenty-four hours). By further way of illustration, addition factors that may affect fatigue level (though not necessarily HRV) include potentially stress-inducing activities such as work and driving in traffic. Such activities may cause a user to become fatigued. The fatigue level may also be determined by comparing the user's measured HRV to a reference HRV, which reference HRV may be based on information gathered from a large number of people from the general public (e.g., similar to the normative statistical data described hereinabove). In another embodiment, the reference HRV is at least partially based on past measurements of the user's HRV (and, e.g., maintained in activity archive module 806).

In additional example implementations of methods 1000 and 1100, fatigue level may be detected periodically (e.g., once every twenty-four hours). This may provide information about the user's fatigue level each day so that the user's activity levels may be tracked precisely. However, the fatigue level may be detected more or less often, as desired by the user (e.g., depending on how accurate/precise the user would like the dynamic activity profile to be) and depending on the specific application/user lifestyle.

Referring now to FIG. 11, one embodiment of method 1100 involves, at operation 1108, creating and updating an activity archive based on the user's activity (e.g., as tracked at operation 1006). The activity archive may include one more memory and/or computing modules or subcomponents thereof, as described in further detail with regard to FIG. 15. As such, the activity archive may store activity information, for example, regarding the user's activity tracked at operation 1006. Activity information may be associated with the user's tracked activity (e.g., such information may be gathered using sensor module 804), including activity type, intensity, duration, and interrelationships thereof (e.g., duration of an activity type at a particular intensity); fatigue level (e.g., including HRV and related data); sleep data; subjective user input; information related to a training load model (including parameters relevant thereto); information related to extern conditions (e.g., weather, altitude, geolocation, time of day, light conditions, etc.); and other information related to internal conditions (e.g., body temperature, cardiac information, breathing, and the like). The activity archive may thus, in some embodiments, act as a source and/or an analysis engine of robust information to generate metrics useful to create personalized, yet realistic combinations of activity types, activity intensities, activity durations, fatigue levels, rest patterns, and so on. Moreover, the activity archive may include other historical information regarding user activity, such as historical information about the user's activity (e.g., information monitored by sensor module 804).

At operation 1010, methods 1000 and 1100 include creating and updating a dynamic activity profile based on the activity profile (created at operation 1004) and the user's activity (e.g., as tracked at operation 1006). The dynamic activity profile may include multiple activity profile data points (or activity parameters) that include, by way of illustration, the activity parameters. For example, the dynamic activity profile may include the user's activity level (e.g., activity type, intensity, and duration), sleep levels (e.g., sleep duration, quality, patterns, etc.), fatigue levels (e.g., including HRV), ongoing subjective feedback from the user, and so on. In one embodiment, the dynamic activity profile is based on one or more statistically manipulated activity parameters that make up at least some of the dynamic activity profile data points. In such an example, creating and updating the dynamic activity profile may entail calculating and maintaining daily averages for any of the above-described activity parameters (e.g., average daily sleep duration). Moreover, a range of high and low values, median values, skew (e.g., shape of distribution of values, including standard deviation, correlations and other statistical relationships between data points, and so on) for each of the activity parameters/data points may also be maintained and incorporated into the dynamic activity profile.

The dynamic activity profile may be created as an initial matter, for example, based on stock or default data or user input and/or after the user activity profile is created (see, e.g., operations 1102 and 1004), but such a profile may be less precise/accurate than if the profile were based on actual, tracked user activity. As such, operation 1010 may involve updating the dynamic activity profile periodically as user activity is tracked (e.g., at operation 1006). In various embodiments, the period for updating the dynamic activity profile may be: (a) selected by the user; (b) default to a stock value (e.g., once daily); (c) set to vary based on changes in the user's activity (e.g., update based on a predetermined amount of variation, as captured in the activity archive); or (d) set to vary based on one or more trigger events (e.g., when HRV is measured, when the user wakes up, etc.). Additionally, the dynamic activity profile may be updated on-the-fly.

One example implementation of the dynamic activity profile includes a matrix stored/maintained in the activity archive (which, by way of example, may be implemented using a memory module). In such an example, the matrix includes one or more data points, cells, etc., each corresponding or associated with an activity parameter. The dynamic activity profile may be accessible to the user, for example, by way of a GUI; may be exportable (e.g., in the form of a .csv file); or may be shared with other users (e.g., via communication medium 704). Furthermore, the dynamic activity profile may be represented graphically, including, by way of illustration, past or projected changes in the dynamic activity profile and/or regarding one or more of the activity parameter values.

In one implementation of the disclosure, the user may be able to manipulate or tune the activity parameters to view/analyze the corresponding effect on the dynamic activity profile. In other words, this aspect of the disclosed systems and methods for creating a dynamic user activity profile may allow a user to view, based on patterns and learned tendencies regarding the user's activity, how changes that the user may make in the user's activity/lifestyle may affect the user going forward. This may provide the user with increased motivation to pursue changes, and may give the user a better understanding of how aggressive the user wants or needs to be to achieve the user's lifestyle goals (e.g., to obtain a desired or planned fitness level). The user may also, for example, view past evolutions of the dynamic activity profile—that is, the user may view freeze-frames of the dynamic activity profile as the dynamic activity profile existed in past time periods, and may call up various activity parameters as tracked during those time period.

As described above, the dynamic activity profile is based on the activity profile and the user's activity. In various embodiments of methods 1000 and 1100, the activity profile contributes to the dynamic activity profile according to a first weighting factor, and the user's activity contributes to the dynamic activity profile according to a second weighting factor. To illustrate, and as described above, the dynamic activity profile may be stored/maintained as a matrix of values. And the values may be categorized according to types of activity parameters (or, for example, types of data points tracked at operation 1006 or calculated thereafter). Further, the values in the dynamic activity profile may include a component attributable to the user input received initially (e.g., at operation 1102) and a component attributable to the activity tracked (or a statistically manipulated version of such tracked activity data point/component). The first weighting factor may be a numerical multiplier that is applied to the user-input component (i.e., the activity profile) and the second weighting factor may be a numerical multiplier that is applied to the tracked-activity component (i.e., the user's activity).

Referring now to FIG. 11, one embodiment of method 1100 includes, at operation 1112, varying the first and second weighting factors. For example, operation 1112 may involve decreasing the first weighting factor as information about the user's activity is tracked and stored in the activity archive. Continuing the example, operation 1112 may further involve increasing the second weighting factor as information about the user's activity is tracked and stored in the activity archive. Varying the first and second weighting factors may, by way of example, increase the accuracy of the dynamic activity profile as more activity information is gathered and as the user's activity tendencies are better learned. The activity profile for the user (e.g., created at operation 1004) is generally based on the user's self-evaluated activity/personal information (e.g., as gathered by the questionnaire described above). Moreover, even when this self-evaluated information is modified according to normative statistical data, the activity profile is still typically a rougher estimate. By contrast, the user's activity (e.g., as represented in the activity archive), may be up-to-date, and the activity archive may analyze patterns and/or tracked tendencies. Therefore, the user's activity may be more tailored to the user, and may be more precise/accurate. More heavily weighting the contribution of the user's activity to the dynamic activity profile may provide the user with better insight into the user's performance, and may better enable the user to set personalized, yet realistic goals and expectations.

In some instances, the first and second weighting factors may be varied to discount the contribution of selected portions of the user activity to the dynamic activity profile. Some portions (e.g., over a period of time, or a particular activity type) may be anomalous in terms of the user's actual activity—as such, incorporating these portions into the dynamic activity profile may decrease the accuracy thereof. By way of illustration, if the user gets sick and needs to rest, the user may refrain from exercise for a time. In instances where such an anomaly represents a departure from the user's normal life, this may be sensed based on the information in the activity archive, and discounted accordingly to avoid spurious results corrupting the dynamic activity profile. Such discounting may, for example, increase the accuracy/legitimacy of the dynamic activity profile, and may also decrease the likelihood that irregularities unrepresentative of the user's lifestyle are incorporated into the dynamic activity profile. As an example, the first and second weighting factors may range from 0 to 1 (e.g., may be decimal/fraction values), and may be complementary, such that the first and second weighting factors add up to 1.

Regarding the accuracy of the dynamic activity profile, one embodiment of method 1100 includes indicating an estimated level of accuracy for the dynamic activity profile. In such an embodiment, the estimated level of accuracy is based on an amount and a consistency of the activity information (e.g., stored/maintained by the activity archive). In typical cases, the accuracy of the dynamic activity profile increases as more and more activity information is captured (e.g., through tracking the user's activity). Further, the accuracy of the dynamic activity profile also typically increases as the activity information captured is consistent. In other words, if there are significant anomalies and otherwise outlying data tracked, this may decrease the accuracy of the dynamic activity profile (in terms of representing the user's typical tendencies). By way of example, the estimated level of accuracy may be represented as a numerical score, as a textual description, graphically, audibly, and so on, and may be provided to the user concurrently with or separately from the dynamic activity profile. In one example implementation, each activity parameter in the dynamic activity profile may be associated with an estimated level of accuracy, depending on how much and/or how consistent the dataset associated with that parameter is.

At operation 1114, one embodiment of method 1100 includes providing an activity recommendation based on one or more of the activity parameters (e.g., as described above with regard to operation 1010). In some instances, the activity recommendation is an early-stage recommendation based on the activity profile (e.g., created at operation 1004, before user activity is tracked). Such an early-stage activity recommendation may be personalized to the extent the same is based on user input (e.g., via the questionnaire) and normative data that approximates the user's activity/tendencies. However, in other instances, the activity recommendation is provided based on tracked activity parameters, and is further based on the dynamic activity profile.

Examples of recommendations for user activity may include activity level (e.g., in terms of type, intensity, and/or duration of activity, and the like), user reaction to activity (e.g., subjective input from user, heart rate, HRV, breathing, etc.), sleep activity (e.g., duration, timing of sleep, conditions/routine related to sleep, etc.), and the like. In a further example, the activity recommendation may be based on a training load model—e.g., to prepare the user to meet a goal, prepare for a race/event, etc., as specified in the model. To expound, the activity recommendation may be based on a training regimen to achieve training goals, for example, to prepare the user for the marathon or other upcoming event. In this example, the activity recommendation may require the user to run a long distance on particular days, and/or to run at a particular pace (or intensity/heart rate) on certain days. In any case, being based (at least in part) on the dynamic activity profile and/or the underlying activity parameters, these activity recommendations may be more personalized/specific to the user, thus being more likely to push the envelope in terms of being both personalized and realistically achievable for the particular user.

By way of further illustration, one embodiment of method 1100 includes recommending an activity level based on the user's past activity, including historical information about user activity type and user activity intensity, duration, and the user's past fatigue levels (associated with past measuring periods, for example). As such, operation 1114 may provide a recommended goal for activity level that is specific to the user's patterns of activity and fatigue, as well as to the user's current level of fatigue (e.g., as determined based on an HRV measurement and/or other factors).

Moreover, when the dynamic activity profile includes contributions from normative data (e.g., based on operation 1004), the recommended activity level may be based on the normative data in addition to the historical data on activity and fatigue. For example, if a fatigue level that is higher than typical (compared to the archive's historical fatigue levels for the user) is detected, operation 1114 may entail recommending an activity level that is lower than typical for the user. In some example implementations, this by done by way of creating a fatigue multiplier. The fatigue multiplier may include, for example, a ratio of the current fatigue level to average historical fatigue level (e.g., as captured in the dynamic activity profile). By contrast, if the fatigue level is lower than typical, operation 1114 may entail recommending an activity level that is higher than typical. In other instances, the activity level is not inversely proportional to the fatigue level—for example, the user's capacity for activity (again, reflected in, for example, the dynamic activity profile and/or the activity archive) may be greater even if the user is more fatigued. Additionally, the normative data may be used to supplement/modify the fatigue modifier, based on what is determined to be statistically typical or average for the user.

In one embodiment, the recommended activity level is based on an anticipation of a future activity, and the future activity is anticipated based on the activity archive and/or the dynamic activity profile. In such an embodiment, it is determined, e.g., from the activity archive, that the user has a higher level of activity than typical (e.g., greater user activity intensity or longer duration of activity types) for a particular day of the week relative to other days of the week. A higher activity level may be recommended for that particular day, due to the learned tendency/pattern of the user's performance. To illustrate, as the user's activity is tracked, it may be determined that the user plays soccer for two hours each Tuesday night. The recommended activity level provided at operation 1114 may be adjusted upward on Tuesdays as a result. In other words, the recommended activity level may conform to the user's desired and/or historical activity levels, having some days as more active and others as less active. In another embodiment, the recommended activity may not conform to the user's schedule if to do so would not help the user perform at the user's peak performance level.

In one case, the activity recommendation is based on an amount of sleep monitored from the previous night. For example, if at operation 1006, eight hours of sleep were tracked for the previous night, a high recommendation for activity level may be provided. This is because the user is likely relatively well rested. In another example, if operation 1006 tracks only four hours of sleep for the user, a lower recommendation for activity level may be provided. This is because the user is likely not as well rested. In another case, the activity recommendation is based on user input that specifies a targeted aggressiveness for achieving a performance goal of the user. For example, the user input may indicate that the user would like to be relatively aggressive in achieving the user's performance/fitness/lifestyle goals. In response, the activity recommendation provided at operation 1114 may include activity levels that are relatively high. This may, for example, push the user to achieve the user's performance goals more quickly.

The activity recommendation, in other instances, is based on the user's learned tendencies (e.g., through the dynamic activity profile and/or the activity archive). To illustrate, the user may tend to be more fatigued on a certain day of the week, to be more fatigued after a certain amount of sleep, or to be more fatigued after a particular level of activity. As more activity information is recorded in the activity archive, method 1100 may involve tracking (e.g., operation 1006), storing/capturing (e.g., operation 1108), and analyzing (e.g., one or more of operations 1108, 1010, 1112, and 1114) developed patterns and interrelationships between the user's activity and fatigue that allow the user's tendencies to be learned. The activity recommendation may then be based on the user's particular, learned tendencies, and may accordingly be tailored specifically for the user to be personalized, yet realistic.

Additionally, method 1100 may entail adjusting the activity recommendation based on one or more of the user's scheduled upcoming activities/events. For example, the activity recommendation may be adjusted for the days or weeks before the user is scheduled to participate in a triathlon, such that the user does not become overworked or underworked before or during the scheduled event. Moreover, in some cases the activity recommendation may be adjusted following a scheduled event. By way of illustration, if the user competes in a scheduled triathlon, the activity recommendation may be adjusted downward (e.g., less activity, more rest/sleep) following the event so that the user can rest and recover. In other instances, the user's tendencies regarding optimum blend of activity, including exercise versus rest, etc., may be learned by way of the above-described operations. In various example implementations of method 1100, operation 1114 is performed by activity recommendation module 902.

Returning briefly to a discussion of the display depicted in FIG. 6, it should be noted that in various embodiments, an activity goal section 603 may display various activity metrics such as a percentage activity goal providing an overview of the status of an activity goal for a timeframe (e.g., day or week), an activity score or other smart activity score associated with the goal, and activities for the measured timeframe (e.g., day or week). For example, the display may provide a user with a current activity score for the day versus a target activity score for the day. Particular methods of calculating activity scores are described in U.S. patent application Ser. No. 14/137,734, filed Dec. 20, 2013, titled “System and Method for Providing a Smart Activity Score”, and which is incorporated herein by reference in its entirety.

In various embodiments, the percentage activity goal may be selected by the user (e.g., by a touch tap) to display to the user an amount of a particular activity (e.g., walking or running) needed to complete the activity goal (e.g., reach 100%). In additional embodiments, activities for the timeframe may be individually selected to display metrics of the selected activity such as points, calories, duration, or some combination thereof. For example, in this particular embodiment activity goal section 603 displays that 100% of the activity goal for the day has been accomplished. Further, activity goal section 603 displays that activities of walking, running, biking, and no activity (sedentary) were performed during the day. This is also displayed as a numerical activity score 5000/5000. In this embodiment, a breakdown of metrics for each activity (e.g., activity points, calories, and duration) for the day may be displayed by selecting the activity.

In various embodiments, a live activity chart 604 may also display an activity trend of the aforementioned metrics (or other metrics) as a dynamic graph at the bottom of the display. For example, the graph may be used to show when user has been most active during the day (e.g., burning the most calories or otherwise engaged in an activity).

In various embodiments, an activity timeline 605 may be displayed as a collapsed bar at the bottom of display 600. In various embodiments, when a user selects activity timeline 605, it may display a more detailed breakdown of daily activity, including, for example, an activity performed at a particular time with associated metrics, total active time for the measuring period, total inactive time for the measuring period, total calories burned for the measuring period, total distance traversed for the measuring period, and other metrics.

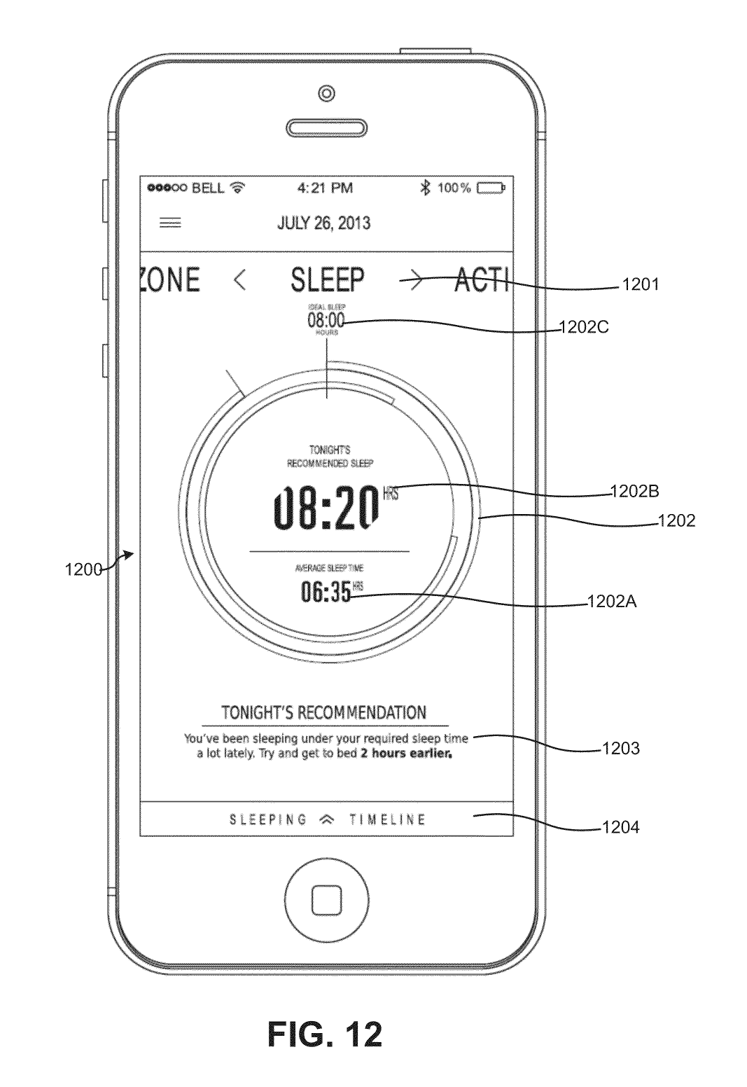

Looking now at further exemplary displays, FIG. 12 illustrates a sleep display 1200 that may be associated with a sleep display module 212. In various embodiments, sleep display 1200 may visually present to a user a record of the user's sleep history and sleep recommendations for the day. It is worth noting that in various embodiments one or more modules of the activity tracking application 210 may automatically determine or estimate when a user is sleeping (and awake) based on an a pre-loaded or learned activity profile for sleep, in accordance with the activity profiles described above. Alternatively, the user may interact with the sleep display 1200 or other display to indicate that the current activity is sleep, enabling the system to better learn that individualized activity profile associated with sleep. The modules may also use data collected from the earphones, including fatigue level and activity score trends, to calculate a recommended amount of sleep. Systems and methods for implementing this functionality are described above with reference to FIGS. 7-11, and also in greater detail in U.S. patent application Ser. No. 14/568,835, filed Dec. 12, 2014, and titled “System and Method for Creating a Dynamic Activity Profile”, and U.S. patent application Ser. No. 14/137,942, filed Dec. 20, 2013, titled “System and Method for Providing an Interpreted Recovery Score,” both of which are incorporated herein by reference in their entirety.

As illustrated, sleep display 1200 may comprise a display navigation area 1201, a center sleep display area 1202, a textual sleep recommendation 1203, and a sleeping detail or timeline 1204. Display navigation area 1201 allows a user to navigate between the various displays associated with modules 211-214 as described above. In this embodiment the sleep display 1200 includes the identification “SLEEP” at the center of the navigation area 1201.

Center sleep display area 1202 may display sleep metrics such as the user's recent average level of sleep or sleep trend 1202A, a recommended amount of sleep for the night 1202B, and an ideal average sleep amount 1202C. In various embodiments, these sleep metrics may be displayed in units of time (e.g., hours and minutes) or other suitable units. Accordingly, a user may compare a recommended sleep level for the user (e.g., metric 1202B) against the user's historical sleep level (e.g., metric 1202A). In one embodiment, the sleep metrics 1202A-902C may be displayed as a pie chart showing the recommended and historical sleep times in different colors. In another embodiment, sleep metrics 1202A-902C may be displayed as a curvilinear graph showing the recommended and historical sleep times as different colored, concentric lines. This particular embodiment is illustrated in example sleep display 1200, which illustrates an inner concentric line for recommended sleep metric 1202B and an outer concentric line for average sleep metric 1202A. In this example, the lines are concentric about a numerical display of the sleep metrics.

In various embodiments, a textual sleep recommendation 1203 may be displayed at the bottom or other location of display 1200 based on the user's recent sleep history. A sleeping detail or timeline 1204 may also be displayed as a collapsed bar at the bottom of sleep display 1200. In various embodiments, when a user selects sleeping detail 1204, it may display a more detailed breakdown of daily sleep metrics, including, for example, total time slept, bedtime, and wake time. In particular implementations of these embodiments, the user may edit the calculated bedtime and wake time. In additional embodiments, the selected sleeping detail 1204 may graphically display a timeline of the user's movements during the sleep hours, thereby providing an indication of how restless or restful the user's sleep is during different times, as well as the user's sleep cycles. For the example, the user's movements may be displayed as a histogram plot charting the frequency and/or intensity of movement during different sleep times.

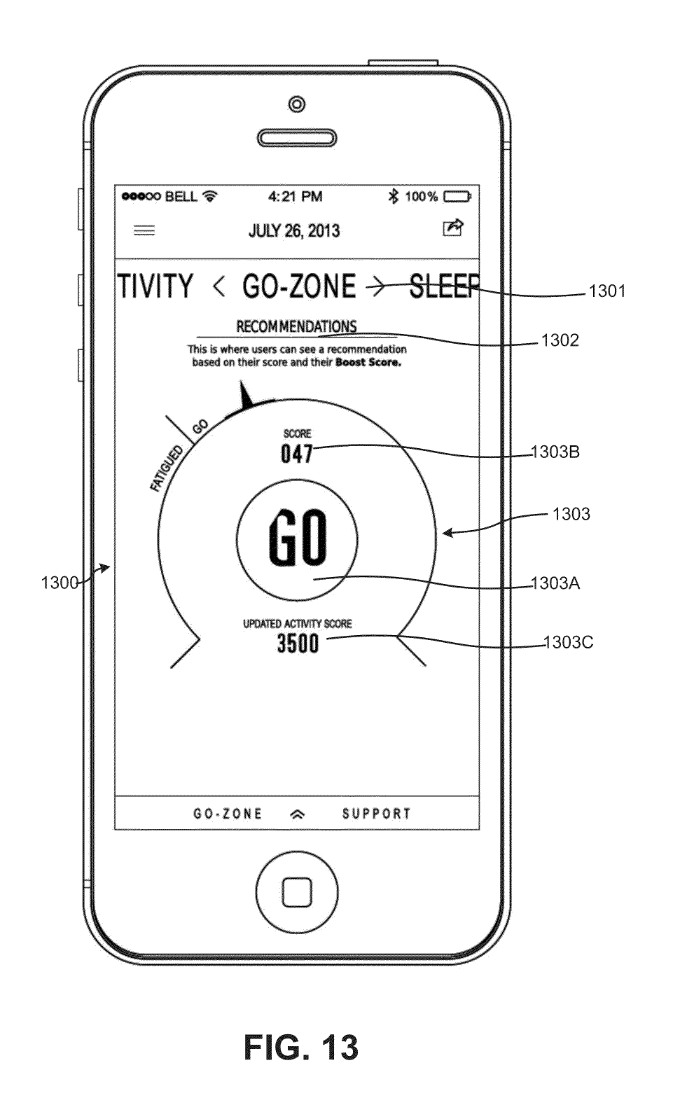

FIG. 13 illustrates an activity recommendation and fatigue level display 1300 that may be associated with an activity recommendation and fatigue level display module 213. In various embodiments, display 1300 may visually present to a user the user's current fatigue level and a recommendation of whether or not engage in activity. It is worth noting that one or more modules of activity tracking application 210 may track fatigue level based on data received from the earphones 100, and make an activity level recommendation. For example, HRV data tracked at regular intervals may be compared with other biometric or biological data to determine how fatigued the user is. Additionally, the HRV data may be compared to pre-loaded or learned fatigue level profiles, as well as a user's specified activity goals. Particular systems and methods for implementing this functionality are described in greater detail in U.S. patent application Ser. No. 14/140,414, filed Dec. 24, 2013, titled “System and Method for Providing an Intelligent Goal Recommendation for Activity Level”, and which is incorporated herein by reference in its entirety.

As illustrated, display 1300 may comprise a display navigation area 1301 (as described above), a textual activity recommendation 1302, and a center fatigue and activity recommendation display 1003. Textual activity recommendation 1302 may, for example, display a recommendation as to whether a user is too fatigued for activity, and thus must rest, or if the user should be active. Center display 1003 may display an indication to a user to be active (or rest) 1303A (e.g., “go”), an overall score 1303B indicating the body's overall readiness for activity, and an activity goal score 1303C indicating an activity goal for the day or other period. In various embodiments, indication 1303A may be displayed as a result of a binary decision—for example, telling the user to be active, or “go”—or on a scaled indicator—for example, a circular dial display showing that a user should be more or less active depending on where a virtual needle is pointing on the dial.

In various embodiments, display 1300 may be generated by measuring the user's HRV at the beginning of the day (e.g., within 30 minutes of waking up.) For example, the user's HRV may be automatically measured using the optical heartrate sensor 122 after the user wears the earphones in a position that generates a good signal as described in method 500. In embodiments, when the user's HRV is being measured, computing device 200 may display any one of the following: an instruction to remain relaxed while the variability in the user's heart signal (i.e., HRV) is being measured, an amount of time remaining until the HRV has been sufficiently measured, and an indication that the user's HRV is detected. After the user's HRV is measured by earphones 100 for a predetermined amount of time (e.g., two minutes), one or more processing modules of computing device 200 may determine the user's fatigue level for the day and a recommended amount of activity for the day. Activity recommendation and fatigue level display 1300 is generated based on this determination.

In further embodiments, the user's HRV may be automatically measured at predetermined intervals throughout the day using optical heartrate sensor 122. In such embodiments, activity recommendation and fatigue level display 1300 may be updated based on the updated HRV received throughout the day. In this manner, the activity recommendations presented to the user may be adjusted throughout the day.

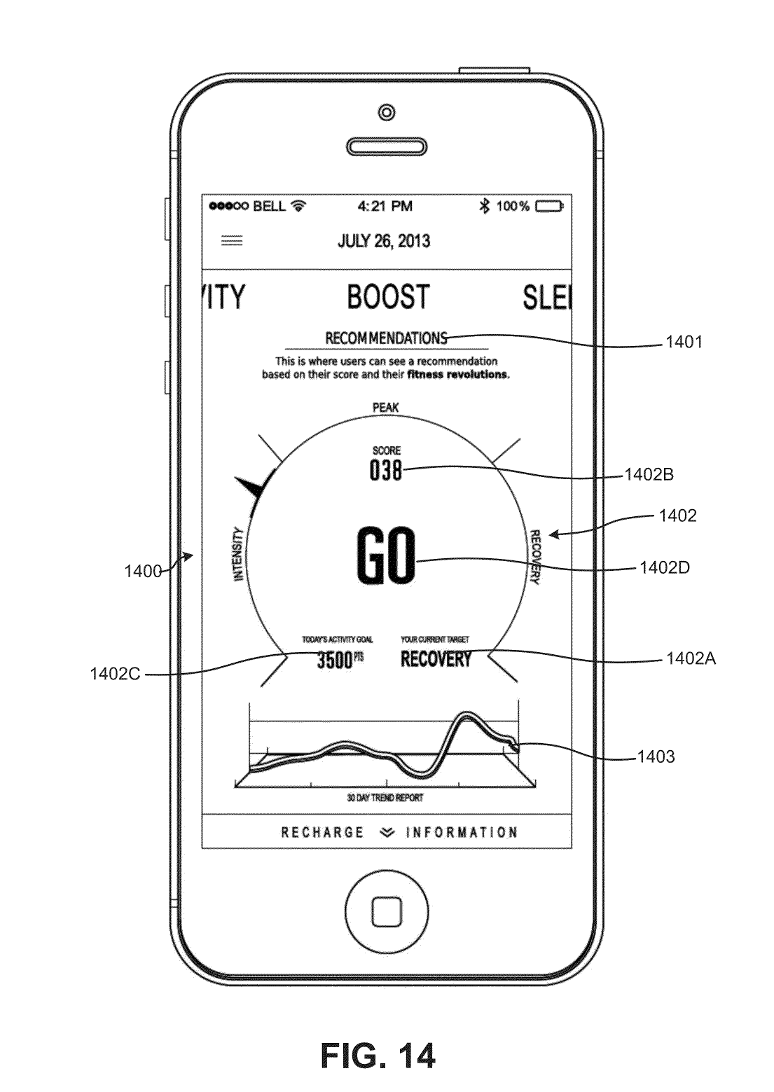

FIG. 14 illustrates a biological data and intensity recommendation display 1400 that may be associated with a biological data and intensity recommendation display module 214. In various embodiments, display 1400 may guide a user of the activity monitoring system through various fitness cycles of high-intensity activity followed by lower-intensity recovery based on the user's body fatigue and recovery level, thereby boosting the user's level of fitness and capacity on each cycle.

As illustrated, display 1400 may include a textual recommendation 1401, a center display 1402, and a historical plot 1403 indicating the user's transition between various fitness cycles. In various embodiments, textual recommendation 1401 may display a current recommended level of activity or training intensity based on current fatigue levels, current activity levels, user goals, pre-loaded profiles, activity scores, smart activity scores, historical trends, and other bio-metrics of interest. Center display 1402 may display a fitness cycle target 1402A (e.g., intensity, peak, fatigue, or recovery), an overall score 1402B indicating the body's overall readiness for activity, an activity goal score 1402C indicating an activity goal for the day or other period, and an indication to a user to be active (or rest) 1402D (e.g., “go”). The data of center display 1402 may be displayed, for example, on a virtual dial, as text, or some combination thereof. In one particular embodiment implementing a dial display, recommended transitions between various fitness cycles (e.g., intensity and recovery) may be indicated by the dial transitioning between predetermined markers.

In various embodiments, display 1400 may display a historical plot 1403 that indicates the user's historical and current transitions between various fitness cycles over a predetermined period of time (e.g., 30 days). The fitness cycles, may include, for example, a fatigue cycle, a performance cycle, and a recovery cycle. Each of these cycles may be associated with a predetermined score range (e.g., overall score 1402B). For example, in one particular implementation a fatigue cycle may be associated with an overall score range of 0 to 33, a performance cycle may be associated with an overall score range of 34 to 66, and a recovery cycle may be associated with an overall score range of 67 to 100. The transitions between the fitness cycles may be demarcated by horizontal lines intersecting the historical plot 1403 at the overall score range boundaries. For example, the illustrated historical plot 1403 includes two horizontal lines intersecting the historical plot. In this example, measurements below the lowest horizontal line indicate a first fitness cycle (e.g., fatigue cycle), measurements between the two horizontal lines indicate a second fitness cycle (e.g., performance cycle), and measurements above the highest horizontal line indicate a third fitness cycle (e.g., recovery cycle).

FIG. 15 illustrates an example computing module that may be used to implement various features of a system for creating and dynamically updating a user activity profile, and that may be used to implement various features of additional systems, apparatus, and methods disclosed herein. One embodiment of the computing module includes a processor, a sensor module, and a memory module. The memory module includes stored computer program code that, along with the processor and the memory module, may be configured to perform a number of operations—one embodiment is as follows. The memory module, the stored computer program code, and the processor are configured to maintain an activity archive that includes activity information. The activity information is received/captured from the sensor module and is representative of the user's activity. The memory module, the stored computer program code, and the processor are further configured to create and update a dynamic activity profile based on initial user input and further based on the activity archive. The initial user input contributes to the dynamic activity profile according to a first weighting factor, and the activity archive contributes to the dynamic profile according to a second weighting factor.

In an additional embodiment of the system for creating and dynamically updating a user activity profile, the memory module, the stored computer program code, and the processor are further configured to vary the first weighting factor based on the activity information in the activity archive, and to vary the second weighting factor based on the activity information in the activity archive. The memory module, the stored computer program code, and the processor, in another embodiment, are further configured to recommend activities for the user based on the dynamic activity profile. Moreover, in various embodiments, the processor, the sensor module, and the memory module, are embodied in a wearable device or other computing device.

In some instances, features of the above-described embodiments of the system for creating and dynamically updating a user activity profile may be substantially similar to those described above with reference to FIGS. 1 through 11 (and the accompanying systems, methods, and apparatus). The example computing module may be implemented and may be used to implement the above-described various features in a variety of ways, as described above with reference to FIGS. 1 through 11, and as will be appreciated by one of ordinary skill in the art upon reading the present disclosure.

As used herein, the term module may describe a given unit of functionality that can be performed in accordance with one or more embodiments of the present application. As used herein, a module may be implemented utilizing any form of hardware, software, or a combination thereof. For example, one or more processors, controllers, ASICs, PLAs, PALs, CPLDs, FPGAs, logical components, software routines or other mechanisms may be implemented to make up a module. In implementation, the various modules described herein may be implemented as discrete modules or the functions and features described can be shared in part or in total among one or more modules. In other words, as would be apparent to one of ordinary skill in the art after reading this description, the various features and functionality described herein may be implemented in any given application and can be implemented in one or more separate or shared modules in various combinations and permutations. Even though various features or elements of functionality may be individually described or claimed as separate modules, one of ordinary skill in the art will understand that these features and functionality can be shared among one or more common software and hardware elements, and such description shall not require or imply that separate hardware or software components are used to implement such features or functionality.

Where components or modules of the application are implemented in whole or in part using software, in one embodiment, these software elements can be implemented to operate with a computing or processing module capable of carrying out the functionality described with respect thereto. One such example computing module is shown in FIG. 15. Various embodiments are described in terms of this example computing module 1500. After reading this description, it will become apparent to a person skilled in the relevant art how to implement the application using other computing modules or architectures.

Referring now to FIG. 15, computing module 1500 may represent, for example, computing or processing capabilities found within desktop, laptop, notebook, and tablet computers; hand-held computing devices (tablets, PDA's, smart phones, cell phones, palmtops, smart-watches, smart-glasses etc.); mainframes, supercomputers, workstations or servers; or any other type of special-purpose or general-purpose computing devices as may be desirable or appropriate for a given application or environment. Computing module 1500 may also represent computing capabilities embedded within or otherwise available to a given device. For example, a computing module may be found in other electronic devices such as, for example, digital cameras, navigation systems, cellular telephones, portable computing devices, modems, routers, WAPs, terminals and other electronic devices that may include some form of processing capability.

Computing module 1500 may include, for example, one or more processors, controllers, control modules, or other processing devices, such as a processor 1504. Processor 1504 may be implemented using a general-purpose or special-purpose processing engine such as, for example, a microprocessor, controller, or other control logic. In the illustrated example, processor 1504 is connected to a bus 1502, although any communication medium can be used to facilitate interaction with other components of computing module 1500 or to communicate externally.

Computing module 1500 may also include one or more memory modules, simply referred to herein as main memory 1508. For example, preferably random access memory (RAM) or other dynamic memory, may be used for storing information and instructions to be executed by processor 1504. Main memory 1508 may also be used for storing temporary variables or other intermediate information during execution of instructions to be executed by processor 1504. Computing module 1500 may likewise include a read only memory (“ROM”) or other static storage device coupled to bus 1502 for storing static information and instructions for processor 1504.

The computing module 1500 may also include one or more various forms of information storage mechanism 1510, which may include, for example, a media drive 1512 and a storage unit interface 1520. The media drive 1512 may include a drive or other mechanism to support fixed or removable storage media 1514. For example, a hard disk drive, a solid state drive, a magnetic tape drive, an optical disk drive, a CD or DVD drive (R or RW), or other removable or fixed media drive may be provided. Accordingly, removable storage media 1514 may include, for example, a hard disk, a solid state drive, magnetic tape, cartridge, optical disk, a CD or DVD, or other fixed or removable medium that is read by, written to or accessed by media drive 1512. As these examples illustrate, removable storage media 1514 can include a computer usable storage medium having stored therein computer software or data.

In alternative embodiments, information storage mechanism 1510 may include other similar instrumentalities for allowing computer programs or other instructions or data to be loaded into computing module 1500. Such instrumentalities may include, for example, a fixed or removable storage unit 1522 and a storage unit interface 1520. Examples of fixed/removable such storage units 1522 and storage unit interfaces 1520 can include a program cartridge and cartridge interface, a removable memory (for example, a flash memory or other removable memory module) and memory slot, a PCMCIA slot and card, and other fixed or removable storage units 1522 and storage unit interfaces 1520 that allow software and data to be transferred from the storage unit 1522 to computing module 1500.

Computing module 1500 may also include a communications interface 1524. Communications interface 1524 may be used to allow software and data to be transferred between computing module 1500 and external devices. Examples of communications interface 1524 may include a modem or softmodem, a network interface (such as an Ethernet, network interface card, WiMedia, IEEE 802.XX or other interface), a communications port (such as for example, a USB port, IR port, RS232 port Bluetooth® interface, or other port), or other communications interface. Software and data transferred via communications interface 1524 may typically be carried on signals, which can be electronic, electromagnetic (which includes optical) or other signals capable of being exchanged by a given communications interface 1524. These signals may be provided to communications interface 1524 via a channel 1528. This channel 1528 may carry signals and may be implemented using a wired or wireless communication medium. Some examples of a channel may include a phone line, a cellular link, an RF link, an optical link, a network interface, a local or wide area network, and other wired or wireless communications channels.

In this document, the terms “computer program medium” and “computer usable medium” are used to generally refer to transitory or non-transitory media such as, for example, main memory 1508, storage unit interface 1520, storage unit 1522, removable storage media 1514, and channel 1528. These and other various forms of computer program media or computer usable media may be involved in carrying one or more sequences of one or more instructions to a processing device for execution. Such instructions embodied on the medium are generally referred to as “computer program code” or a “computer program product” (which may be grouped in the form of computer programs or other groupings). When executed, such instructions may enable the computing module 1500 to perform features or functions of the present application as discussed herein.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of example block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

While various embodiments of the present disclosure have been described above, it should be understood that they have been presented by way of example only, and not of limitation. Likewise, the various diagrams may depict an example architectural or other configuration for the disclosure, which is done to aid in understanding the features and functionality that can be included in the disclosure. The disclosure is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present disclosure. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosure is described above in terms of various example embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present disclosure should not be limited by any of the above-described example embodiments.

What is claimed is:

1. A system for creating and dynamically updating a user activity profile, the system comprising:
an earphone comprising:
a speaker;
a processor;
a heartrate sensor electrically coupled to the processor; and
a motion sensor electrically coupled to the processor, wherein the processor is configured to process electronic input signals from the motion sensor and the heartrate sensor;
a nontransitory computer-readable medium operatively coupled to at least one of one or more processors and having instructions stored thereon that, when executed by at least one of the one or more processors, cause the system to:
create an activity profile for a user;
monitor the user's activity to generate activity information;
maintain an activity archive comprising the activity information; and
update the activity profile based on the activity archive.

2. The system of claim 1, wherein the instructions stored on the one of one or more processors, when executed, further cause the system to: display on a display a recommendation related to the user's activity, wherein the recommendation is based on the activity profile.

3. The system of claim 2, wherein at least one of the activity profile and the recommendation are based on heart rate variability data calculated by one of the one or more processors based on signals received from the heartrate sensor

4. The system of claim 3, wherein the activity information comprises heart rate variability data.

5. The system of claim 3, wherein the activity information is selected from the group consisting of activity level data, sleep data, subjective feedback data, activity level data, and training load data.

6. The system of claim 1, wherein the instructions stored on the one of one or more processors, when executed, further cause the system to: display on a display an indication of an estimated level of accuracy of the activity profile; wherein the estimated level of accuracy is based on an amount and a consistency of the activity information.

7. The system of claim 1, wherein one or more of the nontransitory computer-readable medium and one of one or more processors are embedded in the earphone.

8. The system of claim 1, wherein the heartrate sensor is an optical heartrate sensor protruding from a side of the earphone proximal to an interior side of a user's ear when the earphone is worn, and wherein the optical heartrate sensor is configured to measure the user's blood oxygenation level and to output an electrical signal representative of this measurement to one of the one or more processors.

9. The system of claim 1, further comprising a display wherein a graphical user interface is displayed on the display, the graphical user interface displaying information based on the activity information.

10. The system of claim 9, wherein the information displayed is a recommendation to the user based on the activity information.

11. A method for creating and dynamically updating a user activity profile using earphones with biometric sensors, the method comprising:
creating an activity profile for a user;
tracking the user's activity; and
creating and updating a dynamic activity profile based on the activity profile and the user's activity.

12. The method of claim 11, further comprising receiving user input to an activity questionnaire; wherein the activity profile is based on the user input to the activity questionnaire.

13. The method of claim 12, wherein creating the activity profile further comprises modifying the user input according to normative statistical data.

14. The method of claim 11, further comprising creating and updating an activity archive based on the user's activity.

15. The method of claim 14, wherein the activity profile contributes to the dynamic activity profile according to a first weighting factor, and wherein the user's activity contributes to the dynamic activity profile according to a second weighting factor.

16. The method of claim 15, further comprising decreasing the first weighting factor as information about the user's activity is tracked and stored in the activity archive, and increasing the second weighting factor as information about the user's activity is tracked and stored in the activity archive.

17. The method of claim 11, wherein tracking the user's activity comprises monitoring a movement of the user using a wearable device.

18. The method of claim 17, wherein tracking the user's activity comprises determining an activity level of the user; and wherein the dynamic activity profile is based on one or more of an average of the user's activity level, a range of the user's activity level, and a skew of the user's activity level.

19. The method of claim 11, wherein tracking the user's activity comprises tracking a set of activity parameters; and further comprising providing an activity recommendation based on one or more of the activity parameters.

20. The method of claim 19, wherein the set of activity parameters comprises heart rate variability, sleep duration, sleep quality, subjective feedback from the user, previous activity levels, and training load data.

21. A system for creating and dynamically updating a user activity profile, the system comprising:
an earphone;
a processor;
a sensor; and
a memory comprising a non-transitory computer-readable medium having computer program code stored thereon, wherein the stored computer program code, and the processor are configured to:
maintain an activity archive, the activity archive comprising activity information received from electrical signals generated by the sensor and representative of a user's activity; and
create and update a dynamic activity profile based on initial user input and further based on the activity archive;
wherein the initial user input contributes to the dynamic activity profile according to a first weighting factor, and wherein the activity archive contributes to the dynamic profile according to a second weighting factor.

22. The system of claim 21, wherein the stored computer program code and the processor are further configured to:
vary the first weighting factor based on the activity information maintained in the activity archive; and
vary the second weighting factor based on the activity information in the activity archive.

23. The system of claim 21, wherein the stored computer program code and the processor are further configured to recommend activities for the user based on the dynamic activity profile.

24. The system of claim 21, wherein the processor, the sensor, and the memory module are embodied in the earphone.

25. The system of claim 21, wherein the sensor is a motion sensor.

26. The system of claim 21, wherein the sensor is an optical heartrate sensor protruding from a side of the earphone proximal to an interior side of a user's ear when the earphone is worn, and wherein the optical heartrate sensor is configured to measure the user's blood oxygenation level and to output an electrical signal representative of this measurement to the one or more processors.
